(19) United States
(12) Patent Application Publication
(10) Pub. No.: US 2025/0188422 A1
(43) Pub. Date: Jun. 12, 2025

(54) INDUCED TOTIPOTENT POTENTIAL STEM CELLS, METHODS OF MAKING AND USING

(71) Applicant: Peking University, Beijing (CN)

(72) Inventors: Hongkui Deng, Beijing (CN); Jun Xu, Beijing (CN); Yaxing Xu, Beijing (CN); Jingru Zhao, Beijing (CN)

(21) Appl. No.: 18/845,476

(22) PCT Filed: Jan. 6, 2023
(86) PCT No.: PCT/CN2023/070817

§ 371 Date: Sep. 10, 2024

(30) Foreign Application Priority Data
Mar. 10, 2022 (WO) PCT/CN2022/080149

(51) Int. Cl. C12N 5/074; C12N 5/073

(52) U.S. Cl. CPC C12N 5/0696; C12N 5/0604; C12N 2500/30; C12N 2501/599; C12N 2501/999; C12N 2506/02

(57) ABSTRACT

Factors for deriving totipotent stem cells in vitro that functionally and molecularly resemble cells from totipotent embryos are provided. A cell culture media composition for deriving cell totipotency in vitro of isolated cells and an isolated chemically induced totipotent potential stem cells (ciTPSCs) obtained by using the composition are provided. The composition comprises chemical derivers of totipotency (CDTs) from each of the following groups (1) an HDAC inhibitor, (2) a Dot1L inhibitor, (3) an RARγ agonist, and (4) optionally, a GSK inhibitor, in amounts effective to induce an untreated cell into a totipotent potential stem (TPS) cell. The ciTPSCs can be used in, e.g., cell therapy and tissue engineering.

### CROSS REFERENCE TO RELATED APPLICATION

This application claims priority to PCT Application No. PCT/CN2022/080149, entitled “Induced Totipotent Potential Stem Cells, Methods of Making and Using,” and filed on Mar. 10, 2022, which is incorporated herein by reference.

### FIELD OF THE INVENTION

The invention is generally directed to compositions and methods for deriving totipotent stem cells in vitro that functionally and molecularly resemble cells from totipotent embryos.

### BACKGROUND OF THE INVENTION

During development, early-stage blastomeres are totipotent cells that have the potential to generate an entire individual, including both embryo and extraembryonic components, at the single cell level. The totipotent potential of early-stage blastomeres is gradually restricted at the blastocyst stage, differentiation into epiblast, trophectoderm and primitive endoderm occur. Different self-renewing stem cells, including pluripotent stem cells, trophoblast stem cells and extra-embryonic endoderm cells, can be derived from the blastocyst, which preserve the developmental potentials of epiblast, trophectoderm and primitive endoderm respectively. Importantly, the developmental potentials of these stem cell types are lineage restricted in that they have difficulties in crossing the embryonic and extraembryonic lineage boundaries especially in vivo (Papaioannou, V. E., McBurney, M. W., Gardner, R. L. & Evans, M. J. Fate of teratocarcinoma cells injected into early mouse embryos. Nature 258, 70-73, doi:10.1038/258070a0 (1975); Gardner, R. L. & Rossant, J. Investigation of the fate of 4-5 day post-coitum mouse inner cell mass cells by blastocyst injection. J Embryol Exp Morphol 52, 141-152 (1979); Beddington, R. S. & Robertson, E. J. An assessment of the developmental potential of embryonic stem cells in the midgestation mouse embryo. Development 105, 733-737 (1989)). Compared to these blastocyst-derived stem cell types, totipotent embryonic cells harbor the greatest developmental potency, and there has been a great scientific interest to capture such stem cells from totipotent embryos in vitro, whose unrestricted potency would have broad applications for stem cell biology and regenerative medicine.

Previous studies have shown that a rare 2-cell embryo (2C)-like subpopulation exists in mouse embryonic stem (ES) cell cultures that express 2-cell blastomere molecular markers and have embryonic and extraembryonic developmental potentials (Macfarlan, T. S. et al. Embryonic stem cell potency fluctuates with endogenous retrovirus activity. Nature 487, 57-63, doi:10.1038/nature11244 (2012); Rodriguez-Terrones, D. et al. A molecular roadmap for the emergence of early-embryonic-like cells in culture, Nature genetics 50, 106-119, doi:10.1038/s41588-017-0016-5 (2018)). The major limitation of 2C-like cells is that they are only a minor population from mouse ES cell cultures and reside in a transient intermediate state, which cannot be stabilized in vitro. In recent years, our group and others have shown that small molecule combinations can expand the developmental potentials of pluripotent stem cells toward extraembryonic lineages. Importantly, extended pluripotent stem (EPS) cells can be induced into blastocyst-like structures (WO2017025061A1). Despite their expanded developmental potentials, these cells still are transcriptomically distinct from 2-cell embryos. In addition, their extraembryonic differentiation potentials are still limited when compared with totipotent embryos (Posfai, E. et al. Evaluating totipotency using criteria of increasing stringency. Nature cell biology 23, 49-60, doi:10.1038/s41556-020-00609-2 (2021)). Therefore, it remains a major challenge to capture and maintain bona fide totipotent stem cells in vitro.

It is therefore an object of the present invention to provide totipotent stem cells in vitro.

It is also an object of the present invention to provide compositions for deriving totipotency of isolated pluripotent stem cells in vitro.

It is still an object of the present invention to provide methods of deriving totipotency of isolated pluripotent stem cells in vitro.

It is a further object of the present invention to provide methods of using cells with totipotency.

### SUMMARY OF THE INVENTION

Cocktails of factors which can be used to derive totipotency of isolated cells in vitro have been identified, designated herein as chemical derivers of totipotency (CDTs). CDTs can directly establish totipotent-like stem cells, designated as totipotent potential stem (TPS) cells from embryos such as 2-cell embryos as well as from pluripotent cells such as extended pluripotent stem cells in vitro. The induced totipotent-like stem cells can be stably maintained long term in vitro, with molecular features resembling 2-cell to 4-cell blastomeres. Moreover, they can generate both embryonic and extraembryonic lineages in vivo at the single cell level and form blastocyst-like structures in vitro.

CDTs include: (1) an HDAC inhibitor, (2) a Dot1L inhibitor, (3) an RARγ agonist, and (4) optionally, a GSK inhibitor. In a preferred embodiment, the HDAC inhibitor is an Hdac1 and/or Hdac2 inhibitor, e.g., one selected from VPA (“V”), TSA, MS275, Scriptaid, SAHA, LBH589, FK228, PXD101, Sodium butyrate, LAQ824, CUDC-101, JNJ-26481585, SB939, PCI-24781, ACY-1215, C1994, CUDC-907, RGFP109, Resminostat, Curcumin, Divalproex Sodium, 4-PBA, GSK3117391, CAY10433, CM-675 and MGCD0103; the Dot1L inhibitor is selected from e.g., EPZ004777 (“E”), EPZ5676, and SGC0946; the RARγ agonist is selected from e.g., CD1530 (“D”), AM580, ch55, Palovarotene, CD3254, CD5789, CD437, TTNPB, AGN205327 and RA; the GSK inhibitor is a GSK3 inhibitor, e.g., one selected from CHIR99021 (“C”), AZD2858, LY2090314, BIO, CHIR 98014, SB415286, AZD1080, BRD3731, A 1070722, BIP-135 and SB216763.

Also provided is a method of deriving totipotency of an isolated pluripotent stem cell by culturing a donor cell or a cell derived therefrom using the CDTs disclosed herein. The cell to be induced (i.e., the donor cell) is contacted with the CDTs for a period of time effective to induce totipotency. In a preferred embodiment, cells are cultured in a medium containing the CDTs for a period between 15-150 days, e.g., 15, 30, 45, 60, 75, 90, 105, 120, 135, 150 days.

Also disclosed is an isolated chemically induced totipotent potential stem cell (ciTPSC). An isolated chemically induced totipotent potential stem cell as disclosed herein is identified as a totipotent stem cell based on properties including: i) the cell expression of any one or more, preferably all totipotent marker gene selected from Zscan4, Zfp352, Tcstv1, Tcstv3, MERVL, Dux, Dub1a, Eif1al6, Elf1a9, Gm4340 and Tdpoz4 is present when compared to untreated corresponding cells, optionally after 10 or more passages, and/or ii) the cell expression of any one or more, preferably all pluripotency marker gene selected from Oct4, Nanog and Sox2 is downregulated when compared to untreated corresponding cells. Upregulation or downregulation is determined by comparing the levels of the measured factor in the corresponding cell from which the ciTPSC was obtained. The ciTPSCs disclosed herein can be distinguished from mouse embryo cells, ESCs, or extended pluripotent stem cells at least by the methods that are used to generate them i.e., by their origin. Where mouse embryo cells or ESC are naturally occurring cells, ciTPSCs on the other hand are not naturally occurring, when ciTPSCs are obtained by treating donor cells with a combination of factors as described herein.

The ciTPSCs can be cultured or induced to differentiate into cells of a desired type. The ciTPSCs and their progeny can be used in a number of applications, including but not limited to cell therapy and tissue engineering.

### BRIEF DESCRIPTION OF THE DRAWINGS

FIG. 1. Identification of a chemical cocktail that induce totipotent stem-like cells in vitro.
a. Representative images showing inducing TPS cells from EPS cells under the 4S condition. Scale bar, 100 μm. Similar images were obtained in at least 5 independent experiments.
b. Karyotype analysis of TPS cells after long-term culturing under the 4S condition. Similar images were obtained in 3 independent experiments.
c. Q-PCR analysis of expression of totipotency and pluripotency marker genes in TPS cells converted from EPS cells (EPS-TPS #1) and TPS cells derived from 2-cell embryos (2C-TPS #1 and #2). N=3 biological replicates. Similar results were obtained in at least 2 independent experiments.
d. Heatmap showing the relative expression of representative totipotent and pluripotent genes in TPS and EPS cells.
e. Representative immunofluorescent analysis showing expression of trophectoderm (CDX2) and epiblast (OCT4) markers in TPS derivatives in the chimeric blastocysts. These images showed different focal planes of the same embryo. Scale bar, 20 μm. Td, tdTomato. Similar images were obtained in at least 2 independent experiments.
f. UMAP plot showing the main clusters in the TPS-derived teratomas. Red dotted line indicates extraembryonic cell lineages.
g. Heatmap showing the average expression of representative marker genes for each cluster from the TPS-derived teratomas.
h. Representative images showing chimerism of TPS derivatives in genital ridge of E13.5 embryos. For each image, samples on the upper side were from one chimeric conceptus, and samples on the lower side were from one non-chimeric conceptus. Scale bar, 2 mm. Similar images were obtained in at least 2 independent experiments.
i. Representative morphology of 4S-treated outgrowth derived from 2-cell embryos expressing tdTomato. Scale bar, 100 μm. BF, bright field. Td, tdTomato. Similar images were obtained in at least 2 independent experiments.
j. Representative immunofluorescent analysis of ZSCAN4 expression in TPS cells derived from 2-cell embryos expressing tdTomato. Scale bar, 50 μm. Td, tdTomato. Similar images were obtained in at least 2 independent experiments.

FIG. 2. TPS cells share transcriptomic and epigenetic features with 2-cell blastomere.
a. UMAP plot showing the expression of representative totipotent marker genes in TPS, ES and EPS cells at the single cell level. Different cell types are indicated using different colors.
b. Latent semantic indexing (LSI) analysis comparing developmental progression from zygote to postimplantation (E5.25-E7.5) epiblast with TPS cells, ES cells, EPS cells, TBLCs and spontaneous 2C-like cells. 2C-TPS-sub, TPS 2C-subpopulation. TBLC-sup, subpopulation from TBLCs. The detailed method for constructing the mouse embryonic development trajectory is provided in the method section. Sequencing data of different stem cell types are from GSE33923 (Macfarlan et al., 2012), GSE168728 (Shen et al., 2021), GSE74155 (Chen et al., 2016), and GSE145609 (Posfai et al., 2021).
c. Quadratic programming-based deconvolution analysis showing the transcriptomic similarity between 2-cell embryo and different in vitro cell types. Embryonic cells from different developmental stages are included as the controls. EPS-TPS-sub, TPS 2C-subpopulation from EPS-TPS cells. 2C-TPS-sub, TPS 2C-subpopulation from 2C-TPS cells. TBLC-sup, subpopulation from TBLCs. 2CLC, spontaneous 2C-like cells.
d. ssGSEA analysis showing the similarities between embryonic cells from different developmental stages and in vitro cell types. EPS-TPS-sub, TPS 2C-subpopulation from EPS-TPS cells. 2C-TPS-sub, TPS 2C-subpopulation from 2C-TPS cells. TBLC-sup, subpopulation from TBLCs. 2CLC, spontaneous 2C-like cells.
e. Comparison of regulon activities between TPS 2C-subpopulation (TPS-sub) and EPS cells. Regulons that are enriched in TPS 2C-subpopulation and EPS cells are highlighted.
f. Analysis of regulon activities at different developmental stages. The activities of top 20 regulons that are upregulated and downregulated in TPS 2C-subpopulation (TPS-sub) are quantified.
g. Identification of opened and closed peaks that are enriched in TPS cells. ES and EPS cells were used for comparison. Orange color (high) indicates TPS-enriched opened peaks, and blue color (low) indicates TPS-enriched closed peaks.
h. Average signal intensities of TPS-enriched opened and closed peaks in TPS, ES, EPS cells and 2-cell embryos. Orange color indicates TPS-enriched opened peaks, and blue color indicates TPS-enriched closed peaks.
i. Comparison of global DNA CpG methylation levels in TPS, ES, EPS cells and embryonic cells from different developmental stages (2-cell, 4-cell, E6.5 and E7.5 embryos, E3.5 inner cell mass (ICM)).

FIG. 3. TPS cells can generate both embryonic and extraembryonic lineages in vivo at the single cell level.
a. Representative image showing chimerism of single TPS derivative cells in embryonic and extraembryonic regions in E7.5 conceptuses. Scale bar, 200 μm. BF, bright field. Td, tdTomato. Similar images were obtained in at least 3 independent experiments.
b. Representative immunofluorescent analysis of expression of embryonic (OCT4) and extraembryonic ectoderm (EOMES) markers in chimeric derivative cells from single TPS cells in E7.5 conceptuses. The stained sections were from the same analyzed conceptus. Scale bars, 200 μm. Similar images were obtained in at least 3 independent experiments.
c. Enlarged images of FIG. 3b showing contribution of single TPS derivatives in the extraembryonic ectoderm region. Scale bar, 50 μm.
d. Representative images showing contribution of single TPS derivative cells in E10.5 embryo, yolk sac and placenta. Scale bar, 500 μm. Td, tdTomato. BF, bright field. Similar images were obtained in at least 3 independent experiments.
e. Representative immunofluorescent analysis of E10.5 chimeric placenta generated by injection of single TPS cells (tdTomato labeled). 2-cell embryo-derived TPS cells were used. The left panels show the original images and the right panels show the enlarged images. Anti-TD, anti-tdTomato antibody. Scale bars: left panels, 2 mm; right panels, 20 μm. Similar images were obtained in at least 3 independent experiments.
f. UMAP plot showing the 19 main clusters in E17.5 chimeric placenta. Pink dotted line indicates extraembryonic cell lineages.
g. UMAP plot showing tdTomato expression in E17.5 chimeric placenta. Td+, tdTomato positive cells. Td−, tdTomato negative cells.
h, Heatmap showing the average expression of specific marker genes for each cluster from 3f.

FIG. 4. Induction of blastocyst-like structures from TPS cells in vitro.
a. Representative morphology of induced blastoids from TPS cells. Scale bar, 100 μm. BF, bright field. Td, tdTomato. Similar images were obtained in at least 3 independent experiments.
b. Representative immunofluorescent analysis of trophectoderm (CDX2) and epiblast (OCT4) marker genes in TPS-blastoids. Lower panels show enlarged images of upper panels. Arrows indicates OCT4 and CDX2 expression. Scale bars, upper panels, 100 μm; lower panels, 50 μm. Similar images were obtained in at least 3 independent experiments.
c. UMAP plot showing the expression of representative marker genes for epiblast, trophectoderm, primitive endoderm, and totipotency in TPS-blastoid cells at the single cell level. EPI-like, epiblast-like cells. PE-like, primitive endoderm-like cells. TE-like, trophectoderm-like cells.
d. Heatmap showing expression of 262 representative marker genes for epiblast, primitive endoderm and trophectoderm in TPS blastoid cells. EPI-like, epiblast-like cells. PE-like, primitive endoderm-like cells. TE-like, trophectoderm-like cells.
e. LSI analysis comparing cells from E4.5 blastocysts and TPS-blastoids. Left panel shows cell lineage assignments, and right panel show plots for blastocyst and TPS-blastoids. EPI, epiblast, PE, primitive endoderm. TE, trophectoderm.
f. LSI analysis comparing embryonic cells from different developmental stages with blastoid cells from different studies. The single cell RNA sequencing data of blastoid cells from this study, Rivron et al., 2018 and Sozen et al., 2019 were analyzed. ICM, E3.5 inner cell mass. E4.5, E4.5 epiblast. E5.5, E5.5 epiblast. E6-7, E6-7 epiblast. PE, primitive endoderm from E4.5 to E7.5. TEpre, trophectoderm from E3.5 to E4.5 blastocysts. TEpost, E5.25 to E6.5 extraembryonic ectoderm. EPI-like, epiblast-like cells. PE-like, primitive endoderm-like cells. TE-like, trophectoderm-like cells.
g. ssGSEA analysis showing the similarities between embryonic cells from different developmental stages and blastoid cells from different studies. The single cell RNA sequencing data of blastoid cells from this study, Rivron et al., 2018 and Sozen et al., 2019 were analyzed. SB-EP indicates cells from EPS/TS-blastoids. SB-Lif indicates cells from ES/TS-blastoids.
h. Quadratic programming-based deconvolution analysis showing the transcriptomic similarity between preimplantation trophectoderm and trophectoderm-like cells from different studies.
i. Representative images showing the formation of decidua in the mouse uterus 4 days after TPS-derived blastoids transfer at 2.5 dpc. Scale bar, left image, 5 mm; right image, 500 μm. Similar images were obtained in at least 3 independent experiments.
j. Representative immunofluorescent analysis of PTGS2 expression in TPS-derived decidua. Left scale bar, 500 μm. Right scale bar, 100 μm. Similar images were obtained in at least 2 independent experiments.

FIG. 5. Mechanistic exploration of totipotency induction and maintenance in TPS cells.
a. Western blot analysis showing the levels of histone H3 and H4 acetylation and H3K79me2 of EPS and TPS cells. Similar results were obtained in at least 2 independent experiments.
b. Q-PCR analysis of expression levels of classical RAR downstream target genes in EPS and TPS cells. N=3 biological replicates.
c. Q-PCR analysis of expression levels of representative totipotent marker genes on day 3 upon treatment of different small molecules combinations. In the 4S condition, EPZ004777, VPA, CD1530 and CHIR 99021 were replaced by small molecules target DOT1L, HDAC, RA signaling and GSK3B respectively. N=2 technical replicates. Similar results were obtained in at least 2 independent experiments. EPS, EPS cells. Basal, EPS cells cultured in the basal medium of 4S condition. EPZ rep, VPA rep, CD1530 rep and CHIR rep indicate small molecules that target DOTIL, HDAC, RA and GSK3p respectively.
d. Q-PCR analysis of Hdac1 and Hdac2 expression in EPS cells after shRNA knock down of Hdac1/2. shHdac, Hdac1/2 shRNA. N=2 biological replicates.
e. Q-PCR analysis of expression levels of representative totipotent marker genes on day 3 after knocking down Hdac1/2 in EPS cells during TPS cell induction. EPS, EPS cells, 4S-V, 4S condition without VPA. 4S-V+shHdac, replacement of VPA with Hdac1/2 knock down. N=2 biological replicates.
f. Q-PCR analysis of Dot1l expression in EPS cells after shRNA knock down of Dot1l. shDot1l, Dot1l shRNA. N=3 biological replicates.
g. Q-PCR analysis of expression levels of representative totipotent marker genes on day 3 after knocking down Dot1l in EPS cells during TPS cell induction. EPS, EPS cells, 4S-E, 4S condition without EPZ004777. 4S-V+shDot, replacement of EPZ004777 with Dot1l knock down. N=3 biological replicates.
h. Q-PCR analysis of the effect of inhibiting RAR signaling on totipotency induction (left panel) and maintenance (right panel) in TPS cells. Expression of representative totipotent marker genes were analyzed. RARγi, LY2955303. RARα/βi, LE135. RXRi, UVI3003. N=3 biological replicates.
i. Q-PCR analysis of the effect of 4S chemical cocktail on totipotency maintenance in early preimplantation embryos. Small molecules were treated from the 2-cell embryo stage for 2 days. DMSO was used as the negative control. 2C, 2-cell embryo. N=2 biological replicates.
j. Q-PCR analysis of the effect of knocking down Dux on maintaining totipotency in TPS cells. Expression of representative totipotent marker genes were analyzed. Dux KD, dux knock down. N=2 biological replicates.
k. Q-PCR analysis of the effect of knocking down Dux on inducing totipotency from EPS cells. Expression of representative totipotent marker genes were analyzed. Dux KD, dux knock down. N=2 biological replicates.
l. Q-PCR analysis of the effect of knocking down p53 on inducing totipotency from EPS cells. Expression of representative totipotent marker genes were analyzed. p53 KD, p53 knock down. N=2 biological replicates.

FIG. 6. Identification of small molecules that can increase the percentage of cells expressing totipotent marker genes. a. Representative images showing the effect of exogenous Dux transient overexpression on activating Zscan4 reporter expression in EPS cells on day 3. Scale bar, 200 μm. CTR, control. DUX OE, exogenous Dux overexpression. b. Representative flow cytometry analysis of the effect of exogenous Dux transient overexpression on activating Zscan4 reporter expression in EPS cells on day 3. The percentage of Zscan4-Emerald-GFP positive cells was shown. CTR, control. DUX OE, exogenous Dux overexpression. c. Q-PCR analysis of expression of exogenous Dux (left panel) and totipotent and pluripotent marker genes (right panel) upon exogenous transient overexpression of Dux. exo-Dux, exogenous Dux overexpression. N=2 biological replicates. d. Representative images showing the effect of VPA, EPZ004777, and CD1530 on activating Zscan4 or MERVL reporter expression in EPS cells. Scale bar, 200 μm. V, VPA. E, EPZ004777. CD, CD1530. CTR, N2B27 medium supplemented with CHIR 99021. e-g. Representative flow cytometry analysis of the percentage of Zscan4 or MERVL positive cells in EPS cells on day 3 after treatment of small molecules. For (f), N=2 biological replicates. V, VPA. E, EPZ004777. CD, CD1530. CTR, N2B27 medium supplemented with CHIR 99021. EPS, EPS cells. 4S-3D: EPS cells treated with 4 small molecules (VPA, CHIR 99021, EPZ004777, CD1530) for 3 days. h. Representative images showing the effect of CHIR 99021 on cell proliferation during inducing TPS cells. Scale bar, 200 μm. 4S-C, 4S condition without CHIR 99021. i. Cell number analysis showing that CHIR 99021 promote cell proliferation during the conversion of TPS cells. N=3 biological replicates. 4S-C, 4S condition without CHIR 99021. j. Representative immunofluorescent analysis of ZSCAN4 expression in TPS cells. Scale bar, 200 μm. k. Q-PCR analysis of the expression of totipotent and pluripotent marker genes during inducing TPS cells from EPS cells at different passages. EPS, EPS cells. EPS-TPS-P1/3/5/19: converted cells at passage 1/3/5/19. N=3 biological replicates. 1. Representative images showing sorted Zscan4-Emerald GFP positive cells cultured using the basal medium and 4S-medium. Scale bar, 200 μm. Basal, basal medium of 4S condition. m. Q-PCR analysis of expression levels of representative totipotent and pluripotent marker genes in sorted Zscan4 positive cells cultured in 4S-medium at passage 1 and 5 (4S-P1, 4S-P5). N=2 technical replicates. Similar results were obtained in at least 2 independent experiments. n. Q-PCR analysis of expression levels of representative totipotent and pluripotent marker genes in TPS cells converted from 2i/LIF-ES (mc2i-ES) and LCDM-EPS (mc6-1-EPS) respectively. N=2 technical replicates. Similar results were obtained in at least 2 independent experiments.

FIG. 7. In vitro analysis of extraembryonic developmental potentials of TPS cells. a. Representative immunofluorescent analysis showing the expression of TS markers (CDX2, EOMES, TFAP2C, SOX2) and pluripotency marker OCT4 in TS-like cells. Scale bar, 50 μm. b. Representative immunofluorescent analysis showing the expression of PE markers (GATA6, PDGFRax, SOX7, SOX17) and pluripotency marker OCT4 in PE-like cells. Scale bar, 50 μm.

FIG. 8. Immunofluorescent analysis of blastocysts with chimeric TPS derivatives in vitro. a-c. Representative immunofluorescent images showing contribution of TPS derivatives in trophoectoderm (CDX2, EOMES, CK8, TFAP2C) and primitive endoderm (PDGFRα) in mouse blastocysts. For fluorescent images, the left panels show the original images and the right panels show the enlarged images. BF, Bright field. Td, tdTomato. Scale bars, 20 μm.

FIG. 9. Analysis of the process of deriving TPS cells from 2-cell embryos. Representative immunofluorescent analysis of ZSCAN4 and OCT4 expression in the outgrowth under the 4S condition. Outgrowth cultured under the 2i/LIF condition was used as the control. Scale bar, 100 μm. Similar images were obtained in at least 2 independent experiments.

FIG. 10. Transcriptomic analysis of TPS cells with 2-cell blastomeres and EPS cells. a. Hierarchical clustering analysis of TPS and EPS cell lines. b. Volcano plot showing comparison of transcriptomic differences between TPS and EPS cells. Representative upregulated genes are shown in red plots. c. Gene Ontology analysis of differently expressed genes between TPS and EPS cells. d. Box plots showing the expression of genes upregulated and downregulated in TPS cells at different development stages during preimplantation development (Deng et al., 2014). e. Heatmap showing the expression of 2-cell embryo-specific totipotent signatures in TPS and EPS cells.

FIG. 11. Further analysis of molecular features of TPS cells. a. UMAP plot showing the expression of representative totipotent marker genes in TBLCs at the single cell level. Different cell types are indicated using different colors. PSC, pluripotent stem cells. Meso-like an Endo-like, fibroblast feeders. Intermediate, intermediate cells in the TBLCs. TBLC, TBLC subpopulation highly expressing totipotent marker genes. b. ATAC-seq peaks around representative totipotent and pluripotent marker genes in 2-cell embryos (2-cell), mouse ES cells, EPS cells and TPS cells. c. WGBS analysis of CpG methylation in the loci of representative totipotent marker genes in 2-cell embryo (2-cell), E6.5 and E7.5 embryos (E6.5 and E7.5), EPS cells, TPS cells, and ES cells.

FIG. 12. Further analysis of the chimerism of TPS cells in vivo. a. Representative image showing chimerism of single blastomere of 8-cell embryos expressing EGFP in embryonic and extraembryonic region in E7.5 embryos. Left panel: bright field; right panel: EGFP. Scale bar, 500 μm. Similar images were obtained in at least 3 independent experiments. b. Representative immunofluorescent analysis showing the expression of visceral endoderm marker SOX17 in single TPS derivative cells in E7.5 embryos. Td, tdTomato. Scale bar, 200 m. Similar images were obtained in at least 3 independent experiments. c. Representative images showing contribution of single blastomere of 8-cell embryo expressing tdTomato in E10.5 embryo, yolk sac and placenta. For each image, samples on the left side were from one chimeric conceptus, and samples on the right side were from one non-chimeric conceptus. Scale bars, 5 mm. Similar images were obtained in at least 3 independent experiments. d. Representative flow cytometry analysis of the chimerism of single TPS derivative cells in E10.5 embryo, yolk sac and placenta. Td, tdTomato. WT, wild type. Similar images were obtained in at least 3 independent experiments. e. Representative immunofluorescent analysis of E10.5 chimeric placenta generated by injection of single TPS cell (tdTomato labeled) converted from EPS cells. Anti-TD, antitdTomato antibody. The right panels show enlarged images of the left panels. Scale bars: left panels, 2 mm; right panels, 20 μm. Similar images were obtained in at least 3 independent experiments. f Representative immunofluorescent analysis of E10.5 chimeric placenta generated by injection of single blastomere of 8-cell embryo expressing EGFP. The right panels show enlarged images of the left panels. Scale bars: left panels, 2 mm; right panels, 20 μm. Similar images were obtained in at least 3 independent experiments. g. Representative immunofluorescent analysis of wild type E10.5 placenta. Anti-TD, antitdTomato antibody. The right panels show enlarged images of the left panels. Scale bars: left panels, 2 mm; right panels, 20 μm. Similar images were obtained in at least 3 independent experiments.

FIG. 13. Further analysis of the chimerism in E17.5 conceptuses. a. Representative images showing contribution of single TPS-derived cells (tdTomato labeled) into embryo, yolk sac and placenta in E17.5 mouse conceptuses. BF: bright field. Td, tdTomato. For each image, samples on the left side were from one non-chimeric conceptus, and samples on the right side were from one chimeric conceptus. Scale bars, 10 mm. Similar images were obtained in at least 3 independent experiments. b. Representative flow cytometry analysis of the chimerism of tdTomato positive TPS-derived cells in GFP positive recipient E10.5 placenta. CTR, control. TD, tdTomato. Similar images were obtained in at least 2 independent experiments. c. Representative Q-PCR analysis of trophoblast marker genes expression in tdTomato positive TPS-derived cells in GFP positive recipient E18.5 placenta tissues. N=2 technical replicates. tdTomato positive and GFP negative cells were purified by FACS. GFP positive and tdTomato negative cells were also sorted as the control. Similar results were obtained in at least 2 independent experiments.

FIG. 14. Further mechanistic exploration of totipotency induction and maintenance in TPS cells. a. Average ATAC-seq signal intensities of genomic regions containing RARγ motif in TPS, ES, EPS cells and 2-cell embryos (2cell). b. Q-PCR analysis of expression levels of representative totipotent and pluripotent marker genes on day 3 upon treatment of different small molecules combinations. In the 4S condition, EPZ004777, VPA, CD1530 and CHIR 99021 were replaced by small molecules target DOT1L, HDAC, RA signaling and GSK3D respectively. EPS, EPS cells. Basal, EPS cells cultured in the basal medium of 4S condition. EPZ rep, VPA rep, CD1530 rep and CHIR rep indicate small molecules that target DOT1L, HDAC, RA and GSK3D respectively. N=2 technical replicates. Similar results were obtained in at least 2 independent experiments. c. Western blot analysis showing the protein expression of β-catenin in ES, EPS and TPS cells. ES, ES cells. EPS, EPS cells. 2C-TPS, TPS cells derived from 2-cell embryos. EPS-TPS, TPS cells converted from EPS cells. d. Representative images showing TPS cells can grow without CHIR 99021. 4S-C, 4S condition without CHIR 99021. Scale bar, 50 μm. e. Q-PCR analysis of expression levels of totipotent and pluripotent marker genes in TPS cells cultured in CHIR 0 and CHIR 3 condition. CHIR 0,4S condition without CHIR 99021; CHIR 3, 4S condition. N=2 technical replicates. Similar results were obtained in at least 2 independent experiments.

FIG. 15. Analysis of the role of Oct4 and LIF-stat3 signaling in regulating TPS cells proliferation and totipotent markers expression. a. Q-PCR analysis showing the effect of Oct4 knockout on expression levels of representative totipotent and pluripotent marker genes in TPS cells. Oct4 ko batch 1 and batch 2 indicate different batches of experiments. N=3 biological replicates. b. Representative images showing the effect of Oct4 knockout on TPS cell proliferation. Scale bar, 500 μm. DOX-2D, 2 days after addition of doxorubicin. c. Representative images showing the effect of inhibition of LIF signaling on the proliferation of TPS cells. JAK inhibitor (JAKI) was used. Scale bar, 500 μm. Similar results were obtained in at least 2 independent experiments. d. Q-PCR analysis of expression levels of representative totipotent and pluripotent marker genes in TPS cells upon inhibition of LIF signaling by Jak inhibitors AG490 and Niclosamide. Cells at passage 4 and 6 were analyzed. N=2 technical replicates. Similar results were obtained in at least 2 independent experiments.

FIG. 16. Cell sorting gating strategy. a. Representative gating strategy images of small molecule screening by sorting ZSCAN4 or MERVL positive cells in FIG. 6e. b. Representative gating strategy images of sorting cells derived from Td labeled TPS in chimerism presented on FIG. 12d and FIG. 13b.

### DETAILED DESCRIPTION OF THE INVENTION

Chemical cocktails that enable derivation of novel totipotent cells, termed totipotent potential stem (TPS) cells, with both embryonic and extraembryonic developmental potentials at the single cell level, has been identified. As established by the studies described herein, single TPS cell can contribute to extraembryonic and embryonic tissues at embryonic day 7.5 (E7.5), and E10.5. The studies described here demonstrate TPS cells can be effectively derived in vitro and stably maintained. Thus, TPS cells provide novel cell resources for disease modeling, for example, using humanized animal models studying early development, and generating patient-specific cells for regenerative medicine.

### I. Definitions

The term “cell potency” as used herein a cell's ability to differentiate into other cell types. The more cell types a cell can differentiate into, the greater its potency.

The term “chemically induced totipotent stem cell (“ciTPSC”)” as used herein refers to a totipotent cell with embryonic and extraembryonic developmental potentials at the single cell level in in vivo chimeric assays, by contacting a donor cell with chemical compounds. For example, a ciTPSC derived from human extended pluripotent stem (EPS) cells shows embryonic and extraembryonic developmental potentials at the single cell level following contact with CDTs. Preferably, a ciTPSC as used herein shares features with 2-cell mouse embryos in terms of totipotent markers, transcriptome, chromatin accessibility and DNA methylation patterns and can be induced into blastocyst-like structures resembling preimplantation mouse blastocysts.

The term “corresponding cell” is used to refer to a cell of the same type and from the same organism as the donor cell from which a ciTPSC is obtained. For example, the corresponding cell for a ciTPSC obtained from a mouse embryo cell is a mouse embryo cell which has not been contacted with CDTs.

The term “donor cells” as used herein refers to cells that are to be contacted with the CDTs to induce/confer totipotency.

The term “totipotency” as used herein in connection with a ciTPSC refers to the ability of a ciTPSC to have embryonic and extraembryonic developmental potentials at the single cell level.

The term “epigenetic” as used herein refers to covalent modifications of DNA that are not mutation based, but in some instances can still be passed from generation to generation. Genes that are activated or repressed without any change in DNA sequence are epigenetically controlled. Epigenetic modifications are stable, but potentially reversible alterations in gene expression that occur without permanent changes in DNA sequence. Many types of epigenetic processes have been identified—they include methylation, acetylation, phosphorylation, ubiquitylation, and sumolyation of histones as well as DNA methylation.

The term “induced pluripotent stem cell” (iPSC), as used herein, is a type of pluripotent stem cell artificially derived from a non-pluripotent cell. CiPSCs are also iPSCs; however, they differ from some iPSCs in that they are not genetically engineered to confer pluripotency.

The term “humanized animal model” is used herein to refer to a non-human mammal engrafted with functional human cells or tissues or expressing human transgenes.

“Ability to generate extraembryonic lineages in vivo” as used herein can be determined for example by measuring expression of a trophectoderm marker and/or contribution to both trophectoderm (TE) and ICM (inner cell mass) following microinjection in a chimeric assay as described herein under materials and methods.

The term “isolated” or “purified” when referring to ciTPSCs means chemically induced totipotent stem cells at least 10%, 20%, 30%, 35%, 40%, 45%, 50%, 55%, 60%, 65%, 70%, 75%, 80%, 85%, 90%, 95%, or 99% free of contaminating cell types which are not-totipotent potential stem cells. The isolated TPSCs may also be substantially free of soluble, naturally occurring molecules.

“Media” and “culture medium” as used herein refers to the cell culture milieu. Media is typically an isotonic solution, and can be liquid, gelatinous, or semi-solid, for example, to provide a matrix for cell adhesion or support. Media, as used herein, can include the components for nutritional, chemical, and structural support necessary for culturing a cell.

The term “pluripotency” (or pluripotent), as used herein refers to a stem cell that has the potential to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system). A multipotent stem cell is less plastic and more differentiated, and can become one of several types of cells within a given organ. For example, multipotent blood stem cells can develop into red blood cell progenitors, white blood cells or platelet producing cells. Adult stem cells are multipotent stem cells. Adipose-derived stem cells are multipotent.

“Pluripotent cell” is used herein interchangeably with “pluripotent stem cell”.

The term “small molecule” refers to a molecule, such as an organic or organometallic compound, with a molecular weight of less than 2,000 Daltons, more preferably less than 1, 500 Daltons, most preferably, less than 1,000 Daltons.

### II. Compositions

Cocktails of factors have been identified which can be used to derive totipotency of isolated pluripotent stem cells in vitro. CDTs enable the derivation of totipotent-like stem cells, designated as totipotent potential stem (TPS) cells, that can be stably maintained long-term in vitro. The CDTs can be used to provide at an isolated population of ciTPSC containing least 10%, 20%, 30%, 35%, 40%, 45%, 50%, 55%, 60%, 65%, 70%, 75%, 80%, 85%, 90%, 95%, or 99% free of contaminating cell types such as non-TPS cells.

CDTs include: (1) an HDAC inhibitor, (2) a Dot1L inhibitor, (3) an RARγ agonist, and (4) optionally, a GSK inhibitor. The compositions include CDTs in amounts effective to induce an untreated cell into a totipotent potential stem (TPS) cell. It is within the abilities of one of ordinary skill in the art to determine an equivalent effective concentration for other members within the group of HDAC inhibitor, Dot1L inhibitor, RARγ agonist, and optionally GSK inhibitor based on the effective concentrations disclosed for specific species within the genus, using an in vitro assay.

An optional compound useful in the methods disclosed herein is a GSK inhibitor, for example, a GSK3 inhibitor or one selected from the group consisting of CHIR99021 (“C”), AZD2858, LY2090314, BIO, CHIR 98014, SB415286, AZD1080, BRD3731, A 1070722, BIP-135 and SB216763.

### A. Chemical Derivers of Totipotency

### 1. Small Molecules

Chemical compounds that induce totipotency in vitro, i.e., chemical derivers of totipotency (CDTs) may include small molecules having a molecular weight of less than 2,000 Daltons, more preferably less than 1,500 Daltons, most preferably less than 1,000 Dalton, alone or in combination with proteins. The small molecules may have a molecular weight less than or equal to 900 Daltons or, less than or equal to 500 Daltons. Larger molecules can be used in chemically-induced reprogramming, preferably targeting the same pathway as the small molecules identified here. It is within the abilities of one of ordinary skill in the art to replace, e.g., the HDAC inhibitor, Dot1L inhibitor, RARγ agonist, and GSK inhibitor with a substance having the same or similar function in the signaling pathway (e.g., an shRNA), and such a substance may thus be included as a CDT in some embodiments. In some embodiments, CDTs consist of the listed compounds.

### (i) HDAC Inhibitors

The HDAC inhibitor is preferably an Hdac1 and/or Hdac2 inhibitor, e.g., selected from the group consisting of VPA (“V”), TSA, MS275, Scriptaid, SAHA, LBH589, FK228, PXD101, Sodium butyrate, LAQ824, CUDC-101, JNJ-26481585, SB939, PCI-24781, ACY-1215, CI994, CUDC-907, RGFP109, Resminostat, Curcumin, Divalproex Sodium, 4-PBA, GSK3117391, CAY10433, CM-675 and MGCD0103. VPA may be used in a concentration ranging from 10-1000 μM. For example, the concentration of VPA in the composition can be 10, 50, 100, 200, 300, 400, 500, 600, 700, 800, 900, or 1000 μM.

### (ii) Dot1L Inhibitors

A suitable Dot1L inhibitor is e.g., EPZ004777 (“E”), EPZ5676, and SGC0946. EPZ004777 (“E”) may be used in a concentration ranging from 0.1-10 μM. For example, the concentration of E in the composition can be 0.1, 0.2, 0.5, 1, 1.5, 2, 2.5, 3, 3.5, 4, 4.5, 5, 5.5, 6, 6.5, 7, 7.5, 8, 8.5, 9, 9.5, or 10 μM.

(iii) RARγ Agonists

A suitable Dot1L inhibitor is e.g., CD1530 (“D”), AM580, ch55, Palovarotene, CD3254, CD5789, CD437, TTNPB, AGN205327 and RA. CD1530 (“D”) may be used in a concentration ranging from 0.1-5 μM. For example, the concentration of E in the composition can be 0.1, 0.2, 0.5, 1, 1.5, 2, 2.5, 3, 3.5, 4, 4.5, or 5 μM.

### (iv) GSK Inhibitor

The GSK inhibitor preferably inhibits GSK3 and preferably, is selective for GSK3. A suitable GSK inhibitor is CHIR99021 (“C”), AZD2858, LY2090314, BIO, CHIR 98014, SB415286, AZD1080, BRD3731, A 1070722, BIP-135 and SB216763. The CDT compositions include CHIR99021 in a concentration range from 0.5-10 μM, preferably between 1 and 5 μM, and even more preferably, between 1.5 and 3 μM. For example, the CDTs can include CHIR99021 in concentrations of 1, 1.5, 2, 2.5, 3, 3.5, 4, 4.5, or 5 μM. Concentrations that fall between these numbers are contemplated, as one of ordinary skill in the art can readily fine tune the effective amounts needed.

### B. Cells to be Induced (Donor Cells)

The totipotent stem cells are obtained by culturing isolated embryonic cells or pluripotent cells. In some embodiments, pluripotent cells may be obtained from a mammal such as any mammal (e.g., bovine, ovine, porcine, canine, feline, equine, primate), preferably a human. In some embodiments, the donor cells are obtained from a non-human mammal. Sources include bone marrow, fibroblasts, fetal tissue (e.g., fetal liver tissue), peripheral blood, umbilical cord blood, pancreas, skin or any organ or tissue. In some embodiments, the donor cells may be isolated from a non-human embryo, e.g., a mouse embryo.

In a preferred embodiment the ciTPSCs are obtained from EPS cells. However, other pluripotent cells, for example, embryonic stem cells or induced pluripotent stem cells (iPSCs) may be used, by e.g., firstly being induced into extended pluripotent stem cells (e.g., by a method disclosed in WO2017025061A1), and then the extended pluripotent stem cells are cultured with CDTs. The iPSCs include cells obtained by genetic engineering and/or pure chemical reprograming. In other embodiments, ciTPSCs are obtained from blastocyst.

Preferably, the iPSCs are obtained from chemically induced fibroblasts, adipose-derived stem cells, neural stem cells or cells from the intestinal epithelium. In some embodiment, CiPSCs are obtained from chemically induced neonatal (for example foreskin) or adult fibroblasts. However, iPSCs can be obtained from other cell types including but not limited to: multipotent stem cells, cells of hematological origin, cells of embryonic origin, skin derived cells, fibroblasts, adipose cells, epithelial cells, endothelial cells, mesenchymal cells, parenchymal cells, neurological cells, and connective tissue cells.

Pluripotent cells that can be used in the methods disclosed herein are known in the art and have been described, including methods of maintaining the cells in culture. Mouse embryonic stem (ES) cells are isolated from the inner cell mass of blastocysts, and can be preserved in vitro in a naive inner-cell-mass-like configuration by providing exogenous stimulation with leukaemia inhibitory factor (LIF) and small molecule inhibition of ERK1/ERK2 and GSK3D signaling (termed 2i/LIF conditions). Hallmarks of naive pluripotency include driving Oct4 (also known as Pou5fl) transcription by its distal enhancer, retaining a pre-inactivation X chromosome state, and global reduction in DNA methylation and in H3K27me3 repressive chromatin mark deposition on developmental regulatory gene promoters. Upon withdrawal of 2i/LIF, naive mouse ES cells can drift towards a primed pluripotent state resembling that of the post-implantation epiblast. Although human ES cells share several molecular features with naive mouse ES cells, they also share a variety of epigenetic properties with primed murine epiblast stem cells (EpiSCs). These include predominant use of the proximal enhancer element to maintain OCT4 expression, pronounced tendency for X chromosome inactivation in most female human ES cells, increase in DNA methylation and prominent deposition of H3K27me3 and bivalent domain acquisition on lineage regulatory genes. Derivation of genetically unmodified human naive pluripotent stem cells from already established primed human ES cells, from somatic cells through induced pluripotent stem (iPS) cell reprogramming or directly from blastocysts is disclosed in Gafni, et al., Nature, 504 (7479) 282-286 (2013).

Donor cells may be isolated by disaggregating an appropriate organ or tissue which is to serve as the cell source using techniques known to those skilled in the art. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators.

In some embodiments, the donor cells may include established human ES cells, e.g., commercially available human ES cells. The ciTPSCs obtained by culturing human ES cells in vitro may find use in e.g., regenerative medicine. In some embodiments, the donor cells do not include human ES cells or human embryo cells. In some embodiments, the donor cells are non-human ES cells or non-human embryo cells.

C. Chemically Induced Totipotent Stem Cells (ciTPSCs)

ciTPSCs are identified as a totipotent potential stem cell based on properties including i) the cell expression of any one or more totipotent marker gene selected from Zscan4, Zfp352, Tcstv1, Tcstv3, MERVL, Dux, Dub1a, Eif1al6, Etf1a9, Gm4340 and Tdpoz4 is present when compared to untreated corresponding cells, optionally after 10 or more passages, and/or ii) the cell expression of any one or more pluripotency marker gene selected from Oct4, Nanog and Sox2 is downregulated when compared to untreated corresponding cells; and/or (iii) functionally based on the ability of the cell having embryonic and extraembryonic developmental potentials at the single cell level, and/or being induced into blastocyst-like structures resembling preimplantation mouse blastocysts, and/or sharing features with 2-cell mouse embryos in terms of totipotent markers, transcriptome, chromatin accessibility and DNA methylation patterns, e.g, as illustrated below.

### III. Methods of Making

### A. Induction of Totipotency in Donor Cells

ciTPSCs are produced by contacting cells to be induced (herein donor cells) with culture media containing the CDTs for a sufficient period of time to result in reprograming the cells into chemically induced totipotent stem cell (ciTPSC). A donor cell is contacted with the CDTs disclosed herein in an amount effective to induce and/or reprogram the cell into a totipotent stem cell. One of skill in the art can readily determine the concentrations of the CDT compounds disclosed herein required to provide complete reprograming, by using methods outlined in the examples below, or other methods known in the art.

B. Isolation of ciTPSCs

A substantially purified population of ciTPSCs can be obtained, for example, by extraction (e.g., via density gradient centrifugation and/or flow cytometry) from a culture source. Purity can be measured by any appropriate method. The pluripotent cells can be 99%-100% purified by, for example, flow cytometry (e.g., FACS analysis). Human induced totipotent stem cells can be isolated by, for example, utilizing molecules (e.g., antibodies, antibody derivatives, ligands or Fc-peptide fusion molecules) that bind to a marker or a combination of markers on the induced donor cells and thereby positively selecting cells that bind the molecule (i.e., a positive selection). Other examples of positive selection methods include methods of preferentially promoting the growth of a desired cell type in a mixed population of desired and undesired cell types. Alternatively, by using molecules that bind to markers that are not present on the desired cell type, but that are present on an undesired cell type, the undesired cells containing such markers can be removed from the desired cells (i.e., a negative selection). Other negative selection methods include preferentially killing or inhibiting the growth of an undesired cell type in a mixed population of desired and undesired cell types. Accordingly, by using negative selection, positive selection, or a combination thereof, an enriched population of the cell can be made.

C. Culture and Preservation of ciTPSCs (and their Progeny)

The ciTPSCs can be expanded in culture and stored for later retrieval and use. Once a culture of cells or a mixed culture of stem cells is established, the population of cells is mitotically expanded in vitro by passage to fresh medium as cell density dictates under conditions conducive to cell proliferation, with or without tissue formation. Such culturing methods can include, for example, passaging the cells in culture medium lacking particular growth factors that induce differentiation (e.g., IGF, EGF, FGF, VEGF, and/or other growth factor). Cultured cells can be transferred to fresh medium when sufficient cell density is reached.

In a preferred embodiment, cell culture medium for maintaining ciTPSCs is for example, N2B27 basal medium or 1640 basal medium, supplemented with CDTs disclosed herein. According to some embodiments of the invention, the medium can maintain ciTPSCs 2 to over 50 passages in culture. For example, the medium can maintain ciTPSCs for 2, 3, 4, 5, 6, 7, 8, 9 or 10 passages in culture, preferably, for more than 10 passages, for example for about 20 passages in culture, e.g., for at least about 25, about 30, about 35, about 40, about 45, about 50 passages while in culture. In a preferred embodiment, the ciTPSCs maintain a normal karyotype during the 2, 3, 4, 5, 6, 7, 8, 9, 10, more than 10, for example, about 20 passages in culture.

### IV. Methods of Use

Identification of a readily available source of totipotent stem cells that can give rise to a desired cell type or morphology is important for therapeutic treatments, tissue engineering and research. The availability of totipotent stem cells would be extremely useful in transplantation, tissue engineering, regulation of angiogenesis, vasculogenesis, organ regeneration, humanized animal models, cell replacement or cell therapies as well as the prevention of diseases, etc. Such stem cells can also be used to introduce a gene into a subject as part of a gene therapy regimen.

### A. Providing Differentiated Somatic Cells (Re-Differentiated Cells)

Once established, a culture of totipotent stem cells may be used to produce progeny cells, for example, fibroblasts capable of producing new tissue. The ciTPSCs can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells, blood cells, retinal cells (and other cells involved in sensory perception, such as those that form hair cells in the ear or taste buds on the tongue), and nervous tissue including nerves. The re-differentiated cells can be can be expanded in culture and stored for later retrieval and use.

### B. Cell Therapy

Therapeutic uses of the derived totipotent potential stem cells, include transplanting the totipotent potential stem cells, cell populations, or progeny thereof into individuals to treat a variety of pathological states including diseases and disorders resulting from cancers, wounds, neoplasms, injury, viral infections, diabetes and the like. Treatment may entail the use of the cells to produce new tissue, and the use of the tissue thus produced, according to any method presently known in the art. The cells may be implanted, injected or otherwise administered directly to the site of tissue damage so that they will produce new tissue in vivo. In one embodiment, administration includes the administration of genetically modified ciTPSCs or their progeny.

In a preferred embodiment, the ciTPSCs are obtained from autologous cells i.e., the donor cells are autologous. However, the cells can be obtained from heterologous cells. In one embodiment, the donor cells are obtained from a donor genetically related to the recipient. In another embodiment, donor cells are obtained from a donor genetically un-related to the recipient. If the human ciTPSCs are derived from a heterologous (non-autologous/allogenic) source compared to the recipient subject, concomitant immunosuppression therapy is typically administered, e.g., administration of the immunosuppressive agent cyclosporine or FK506. However, due to the immature state of the human induced donor cells such immunosuppressive therapy may not be required. Accordingly, in one embodiment, the human induced donor cells can be administered to a recipient in the absence of immunomodulatory (e.g., immunsuppressive) therapy. Alternatively, the cells can be encapsulated in a membrane, which permits exchange of fluids but prevents cell/cell contact. Transplantation of microencapsulated cells is known in the art, e.g., Balladur et al., Surgery, 117: 189-94, 1995; and Dixit et al., Cell Transplantation 1: 275-79 (1992). ciTPSCs and their progeny can be used to make tissue engineered constructions, using methods known in the art. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. Tissue engineering technology frequently involves selection of an appropriate culture substrate to sustain and promote tissue growth. In general, these substrates should be three-dimensional and should be processable to form scaffolds of a desired shape for the tissue of interest.

The ciTPSCs can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells (e.g., alpha cells, delta cells, PP cells, and beta cells), blood cells (e.g., leukocytes, erythrocytes, macrophages, and lymphocytes), retinal cells (and other cells involved in sensory perception, such as those that form hair cells in the ear or taste buds on the tongue), and nervous tissue including nerves.

The ciTPSCs can be formulated for administration, delivery or contacting with a subject, tissue or cell to promote de-differentiation in vivo or in vitro/ex vivo. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

The induced pluripotent cells can be administered to a patient by way of a composition that includes a population of ciTPSCs or ciTPSC progeny alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer.

### C. Animal Models and Organ Regeneration

Isolated ciTPSCs can be used to generate animal models incorporating ciTPSCs from a desired species (donor) into a second animal (recipient) of the same or different species. The donor animal can be a mammal such as a human, mouse, rat, pig, cattle, sheep, goat, horse, dog, chimpanzee, gorilla, orangutan, monkey, marmoset, etc. In some preferred embodiments, the donor mammal is a human and the recipient mammal is non-human, used to provide a humanized animal model. In other embodiments, the donor and recipient animals are size matched. The recipient may be any animal other than human, such as pig, rat, mouse, cattle, sheep, goat, horse, dog, chimpanzee, gorilla, orangutan, monkey, marmoset, and bonobo. The ciTPSCs can be used for organ regeneration in a mammal, which is not a human; ciTPSCs can be used to produce a desired organ in the mammal where the mammal has an abnormality associated with a lack of development of that organ in a development stage.

The method includes transplanting ciTPSCs into a blastocyst stage fertilized egg of the recipient non-human mammal; developing the fertilized egg in a womb of a non-human surrogate parent mammal to obtain a litter, and obtaining the organ from the litter, using methods known in the art. Examples of organs that can be produced include, but are not limited to, solid organ with a fixed shape, such as kidney, heart, pancreas, cerebellum, lung, thyroid gland, hair, and thymus. The recipient embryo may be from any animal other than human, such as pig, rat, mouse, cattle, sheep, goat, horse, dog, chimpanzee, gorilla, orangutan, monkey, marmoset, etc.

Methods for generating humanized mouse models are known in the art. Examples of recipient embryos having an abnormality associated with the development of an organ of interest, and which can be used to regenerated that organ include, Sall1 knockout animal having an abnormality associated with a lack of development of a kidney in the development stage (Nishinakamura, et al., Development, 128: 3105-3115 (2001); a Pdx1 knockout animal having an abnormality associated with a lack of development of a pancreas in the development stage (Offield, et al., Development, 122: 983-995 (1996); a Wnt-1 (int-1) knockout animal having an abnormality associated with a lack of development of a cerebellum in the development stage (McMahon, et al., Cell, 62: 1073-1085, (1990); a T/ebp knockout animal having an abnormality associated with a lack of development of a lung and a thyroid gland in the development stage (Kimura, et al., Genes and Development, 10: 60-69, 1996); or a dominant negative-type transgenic mutant animal model which overexpresses the deficiency of an intracellular domain of fibroblast growth factor (FGF) receptor (FGFR), and which causes deficiencies of multiple organs such as kidney and lung (Celli, et al., EMBO J., 17: 1642-655, (1998)), can be used. Alternatively, nude mice can be used to produce of hair or thymus.

### IV. KITS

Kits are provided which include the chemical inducers of totipotency (CDTs) disclosed herein. The CDTs are as described above. These may be in a form having defined concentrations to facilitate addition to cell culture media to produce a desired concentration. The kit may include directions providing desired concentration ranges and times of administration based on the donor cell types. The kit may also include cell culture media which is pre-mixed with the CDTs for culture of donor cells to induce totipotency.

The present invention will be further understood by reference to the following non-limiting examples.

### EXAMPLES

### Methods

### Mice

The mouse strain B6-Tg (C57BL/6-td Tomato) was bought from Beijing Vitalstar Biotechnology Co., Ltd. Other mouse strains ICR and EGFP were purchased from Peking University Health Science Center Department of Laboratory Animal Science. All animal experiments were performed in accordance with the NIH guidelines. And all mice experiments were approved by the Institutional Animal Care and Use Committee of Peking University. The mice were housed in a temperature control room (22±1° C.) with 40-60% humidity, under a 12-h light/dark cycle between 06:00 and 18:00.

### Culture of Mouse TPS Cells

All cell lines were cultured under 20% 02 and 5% C02 at 37° C. Mouse TPS cells were cultured in 4S medium contained N2B27 basal medium [50% DMEM/F12 (Gibco, 11330-032), 50% Neurobasal (Gibco, 21103-049), 0.5% N2 supplement (Gibco, 17502-048), 1% B27 supplement (Gibco, 12587-010), 1% GlutaMAX (Gibco, 35050-061), 1% nonessential amino acids (Gibco, 11140-050)] which promoted cell proliferation, or 1640 basal medium [RPMI Medium 1640 basic (Gibco, 22400-089), 0.5% N2 supplement (Gibco, 17502-048), 1% B27 supplement (Gibco, 12587-010), 1% GlutaMAX (Gibco, 35050-061), 1% nonessential amino acids (Gibco, 11140-050)] which enhanced totipotent molecular features, supplemented with 5% knockout serum replacement (Gibco, 10828-028), 0.2% sodium pyruvate (Gibco, 11360-070), 0.14% sodium DL-lactate solution (Sigma, L7900) and 0.1% Chemically Defined Lipid Concentrate (Gibco, 11905-031), VPA (50 μM; Selleck, S3944), CHIR-99021 (3 μM; Selleck, S1263), EPZ004777 (1 μM, Selleck, S7353) and CD1530 (0.2 M-0.5 μM; Tocris, 2554). Mouse TPS cells were cultured on mitomycin C inactivated mouse embryonic fibroblast cells (feeders, 3*10⁴ cells per cm²). The culture medium was changed every day. TPS cells were passaged every three days with 0.05% trypsin-EDTA (Gibco, 25300-062) and seeded at a split ratio ranging from 1:3 to 1:10. To generate TPS cells, EPS cells or ES cells were dissociated and cultured on feeders in 4S medium for the first three passages, EPS cells or ES cells need to seed at a lower split ratio 1:3 to improve cell viability. After five passages, TPS cells were generated for further experiments.

Derivation of Mouse TPS Cells from 2-Cell Embryos

TPS cells were derived from 2-cell embryos of B6-Tg mice. For the 2-cell embryos, the zona pellucida was removed by Acidic Tyrode's Solution (Sigma, T1788). When the zona pellucida disappeared, the 2-cell embryos were washed three times by M2 Medium (Sigma, M7167), and then, they were seeded on feeders in 4S medium. 2-3 days later, the fresh 4S medium was changed. After 6 days, outgrowths were picked and dissected into small clumps. 4 days later, outgrowths were picked again and digested by 0.05% trypsin-EDTA. Single cells were seeded on feeder in 4S medium supplemented with Y27632 (10 μM; Tocris, 1254). TPS colonies emerged gradually. During the first five passages, it is recommended to seed the cells in 4S medium supplemented with Y27632. The second day, Y27632 was removed. The newly established cell lines were passaged using 0.05% trypsin-EDTA every three days, and used for further analysis.

Derivation of TPS Cells from EPS Cells

All cell lines were cultured under 20% O₂ and 5% CO₂ at 37° C. Mouse EPS cells and ES cells were cultured on feeders (3*10⁴ cells per cm²) in N2B27-LCDM medium or N2B27-2i/LIF medium. N2B27 medium was prepared by including: 50% DMEM/F12 (Gibco, 11330-032), 50% Neurobasal (Gibco, 21103-049), 0.5% N2 supplement (Gibco, 17502-048), 1% B27 supplement (Gibco, 12587-010), 1% GlutaMAX (Gibco, 35050-061), 1% nonessential amino acids (Gibco, 11140-050). N2B27-LCDM medium was prepared by including: recombinant human LIF (10 ng/ml, peprotech, 300-05), CHIR-99021 (3 μM; Selleck, S1263), (S)-(+)-Dimethindene maleate (2 μM, Tocris, 1425), Minocycline hydrochloride (2 μM, Tocris, 3268). N2B27-2i/LIF medium was prepared by including: recombinant human LIF (10 ng/ml), CHIR99021 (3 μM), PD0325901 (1 μM, Selleck, S1036).

Mouse EPS cells and ES cells cultured in N2B27-LCDM medium or N2B27-2i/LIF medium for two days were used for TPS generation. Mouse EPS cells and ES cells were washed with DMEM/F12, then 0.05% trypsin-EDTA (Gibco, 25300-062) was added and incubated for 3 min, then DMEM (Gibco, 11965092) supplemented with 10% FBS (Hyclone, SH30070.03) was added. The cells were pipetted up and down several times into single cells. Cells were centrifuged and re-suspended in 4S medium contained N2B27 which promoted cell proliferation or RPMI Medium 1640 basic (Gibco, 22400-089) which enhanced totipotent molecular features supplemented with 5% knockout serum replacement (Gibco, 10828-028), 0.2% sodium pyruvate (Gibco, 11360-070), 0.14% sodium DL-lactate solution (Sigma, L7900) and 0.1% Chemically Defined Lipid Concentrate (Gibco, 11905-031), VPA (50 μM; Selleck, S3944), CHIR-99021, EPZ004777 (1 μM, Selleck, S7353) and CD1530 (0.2 M-0.5 μM; Tocris, 2554). EPS cells and ES cells were seeded at a split ratio 1:10 on feeders in 4S medium. The 4S medium was changed every day. TPS cells were passaged every three days with 0.05% trypsin-EDTA and seeded at a split ratio ranging from 1:3 to 1:10. For the first three passages, TPS cells need to passage at a high density (1:3) to improve cell viability. After five passages, TPS cells gradually proliferate well in 4S medium and were generated for future experiments.

### Transient Overexpression of Exogenous Dux in Zscan4-Emerald GFP and MERVL-tdTomato Reporter Cells

Plasmid containing exogenous Dux driven by CMV promoter was transfected into Zscan4-Emerald GFP and MERVL-tdTomato reporter cells by nucleofection. LCDM medium was used for culturing the cells after the transfection. Cells were harvested for further analysis after 3 days of transfection.

### Flow Cytometry

Cells were gently digested into single cells using 0.05% trypsin-EDTA. Suspensions were filtered through a 40 m cell strainer. Then, the samples were analyzed on CytoFLEX (Beckman Coulter). Data analysis was performed using CytExpert software.

### Cell Sorting and Culture of Sorted Cells

Cell sorting was performed on BD FACSAria SORP. MERVL-tdTomato positive or Zscan4-Emerald GFP positive cells were sorted and seeded on feeder with TPS medium or basal medium at density of 2*10⁴ cells per cm². The culture medium was changed every day. Sorted cells were passaged every three days with 0.05% trypsin-EDTA (Gibco, 25300-062) and seeded at a split ratio ranging from 1:3 to 1:10.

### Karyotype Analysis

Cells were prepared to give a 50±70% confluence on day of sampling. After 2 h incubation with fresh medium, a Colcemid solution was added to the medium at a final concentration of 0.02 mg/ml and incubated for 1 h. Then the cells were washed in PBS, trypsinized and spun down. To obtain a single cell suspension, the pellet was re-suspended in hypotonic solution (0.56% KCl), and left at room temperature for 6 min. After spinning and removing hypotonic solution, 5 mL of ice-cold fixative (3:1 methanol: acetic acid) was added dropwise to the suspension, left at room temperature for 5 min and then spun down. The fixing procedure was further repeated for additional three times. Finally, the pellet was re-suspended in a final volume of 1 mL fixative. The cells were then dropped onto 5% acetic acid ±ethanol (ice-cold) washed slides and stained with Giemsa. For each analysis, at least 30±40 metaphases were examined. The number of chromosomes as well as the presence of structural chromosomal abnormalities was examined.

### Quantitative PCR Analysis

The total RNAs were isolated using the Direct-zol RNA Kits (ZYMO Research, R2052). RNA was converted to cDNA using TransScript FirstStrand cDNA Synthesis SuperMix (TransGen Biotech, AT311). Quantitative PCR analysis was conducted using the KAPA SYBR FAST qPCR Kit (KAPA Biosystems, KK4601) with the Bio RAD CFX Connect Real-Time System. The primers that were used for Q-PCR analysis are listed in Table 1. The data were analyzed using the delta-delta CT method.

TABLE 1-1
qPCR primers used in this study

| Gene | Primer name | Sequence (5′ to 3′) |
|---|---|---|
| MuERV-c | MERVL_c_F | GGTGGTCGAGATGGAGGTTA |
| | MERVL_c_R | ATGAAGGAAGTGGTCCAACG |
| MuERV-d | MERVL_d_F | ACAGCCTAATCCAAGCAGGA |
| | MERVL_d_R | ATCCTCGTTTCTGCAACTGG |
| Zscan4 | Zscan4-1-F | GAGATTCATGGAGAGTCTGACTGA |
| | | TGAGTG |
| | Zscan4-1-R | GCTGTTGTTTCAAAAGCTTGATGA |
| | | CTTC |
| Zfp352 | ZFP352-1-F | AAGTCCCACATCTGAAGAAACAC |
| | ZFP352-1-R | GGGTATGAGGATTCACCCACA |
| Tcstv1 | Tcstv1-F | TGAACCCTGATGCCTGCTAAGACT |
| | Tcstv1-R | AGATGGCTGCAAAGACACAACTGC |
| Tcstv3 | Tcstv3-F | AGAAAGGGCTGGAACTTGTGACCT |
| | Tcstv3-R | AAAGCTCTTTGAAGCCATGCCCAG |
| Nanog | Nanog-1-F | TCTTCCTGGTCCCCACAGTTT |
| | Nanog-1-R | GCAAGAATAGTTCTCGGGATGAA |
| Oct4 | Oct3/4-up | TGAGGAGGGATTAAAAGCACA |
| | Oct3/4-dn | CAAAATGATGAGTGACAGACAGG |
| Sox2 | mSox2-up | AAACCACCAATCCCATCCA |
| | mSox2-dn | CCCCAAAAAGAAGTCCCAAG |
| Cdx2 | cdx2-2-F | AAACCTGTGCGAGTGGATG |
| | cdx2-2-R | TCTGTGTACACCACCCGGTA |
| Krt8 | Krt8-q-F-1 | GTCCATCAGGGTGACTCAGAAATC |
| | Krt8-q-R-1 | GAAAAGCTGGAAGAGCTGATGC |
| β-actin | mActinb_fwd | TAGGCACCAGGGTGTGATGG |
| | mActinb_rev | CATGGCTGGGGTGTTGAAGG |
| PL2 | q-pl2-F2 | CCAACGTGTGATTGTGGTGTC |
| | q-pl2-R2 | CAGGCCATAGGTCCAAGCTG |
| GCM1 | q-gcm1-F2 | CCTCCAACTCCTTACGGATGA |
| | q-gcm1-R2 | CCTCCAACTCCTTACGGATGA |
| Hand1 | q-Hand1-F2 | CTACCAGTTACATCGCCTACTTG |
| | q-Hand1-R2 | ACCACCATCCGTCTTTTTGAG |
| Tpbpa | q-Tpbpa-F2 | CACAGTAGCGAAAATGACCAGG |
| | q-Tpbpa-R2 | TCCTCCTCTTCAAACATTGGGT |
| Ctsq | q-Ctsq-F2 | CATTGCCAGTTGACAACACAAG |
| | q-Ctsq-R2 | ATAGCCTTCATTTCGCCAATCA |
| PLF | q-plf-F1 | TCCTGGATACTGCTCCTACTACT |
| | q-plf-R1 | GACCATTCCTCATTGCACACA |
| Dux | Dux-1-F | CCCAGCGACTCAAACTCCTTC |
| | Dux-1-R | GGACTTCGTCCAGCAGTTGAT |
| Dub1a | Dub1a-up | GGTCTCAGATCAAGTGTCACCAC |
| | Dub1a-dn | ATTCTCCCCACATAGCTCTTCTG |
| Eif1al6 | Eif1al6-F | CAACACAATTGGCATCTTGACG |
| | Eif1al6-R | GCATTGACAAAACACACTCATGC |
| Eif1al9 | Eif1al9-F | CAATGCAGGTAGTTGTTAAGAGCA |
| | Eif1al9-R | GAAAATGGCTTAACACAAAGGACA |
| Gm4340 | GM4340-up | AATCGAGGCACTGGGTCTAAG |
| | GM4340-dn | GCCGCCCCTATTTTTGCTTTG |
| Tdpoz4 | Tdpoz4-up | GATGATGGGCTTCATCTACACAG |
| | Tdpoz4-dn | CTCACACATGACCATCAAGTCCT |
| exo-Dux | DUX-synthesis- | ATCCCGGTAGCATGGAGCAT |
| | q-F-1 | |
| | DUX-synthesis- | CACTTGTGGGGGTTGTGACTTT |
| | q-R-1 | |
| Cebpa | q-Cebpa-F | CAAGAACAGCAACGAGTACCG |
| | q-Cebpa-R | GTCACTGGTCAACTCCAGCAC |
| Cyp26a1 | q-Cyp26a1-F1 | AAGCTCTGGGACCTGTACTGT |
| | q-Cyp26a1-R1 | CTCCGCTGAAGCACCATCT |
| Hoxa1 | q-hoxa1-F2 | CCCAGACGGCTACTTACCAGA |
| | q-hoxa1-R2 | CATAAGGCGCACTGAAGTTCT |
| Hoxa5 | q-hoxa5-F | CTCATTTTGCGGTCGCTATCC |
| | q-hoxa5-R | ATCCATGCCATTGTAGCCGTA |
| Hoxb1 | q-hoxb1-F | GCCCCAACCTCTTTTCCCC |
| | q-hoxb1-R | GACAGGATACCCCGAGTTTTG |
| Rarb | q-rarb-F | CTGCTCAATCCATCGAGACAC |
| | q-rarb-R | CTTGTCCTGGCAAACGAAGC |
| Stra8 | q-Stra8-F1 | ACAACCTAAGGAAGGCAGTTTAC |
| | q-Stra8-R1 | GACCTCCTCTAAGCTGTTGGG |
| Hdac1 | Q-Hdac1-F-1 | AGTCTGTTACTACTACGACGGG |
| | Q-Hdac1-R-1 | TGAGCAGCAAATTGTGAGTCAT |
| Hdac2 | Q-Hdac2-F-3 | CGGATGACTCATAACTTGCTGC |
| | Q-Hdac2-R-3 | TTCTCCGACGTTAAATCTCTGC |
| Dot1l | Q-Dot1l-F-3 | CAACTGCAAACATCACTACGGA |
| | Q-Dot1l-R-3 | TCACCTCGTTCCAGTGTGTAT |
| p53 | p53-F | GTCACAGCACATGACGGAGG |
| | p53-R | TCTTCCAGATGCTCGGGATAC |

TABLE 1-2
Oligonucleotides used in this study
Cloning sgRNA in px330

| Dux sgRNA 1#_fwd | AAGGCACACAGCCGCTTGCT |
|---|---|
| Dux sgRNA 1#_rev | AGCAAGCGGCTGTGTGCCTT |
| Dux sgRNA 1#_fwd | GACTTTCCCCACTAGTGGCT |
| Dux sgRNA 2#_rev | AGCCACTAGTGGGGAAAGTC |
| p53 sgRNA 1#_fwd | GAACAGATCGTCCATGCAGTG |
| p53 sgRNA 2#_rev | CACTGCATGGACGATCTGTTC |
| p53 sgRNA 1#_fwd | GTCCACCCGGATAAGATGCTG |
| p53 sgRNA 2#_rev | CAGCATCTTATCCGGGTGGAC |
| shRNA sequence | |
| in TRC1.5 | |
| Vector (pLKO.1- | |
| puro Vector) | |
| Hdac1 shRNA 1# | CCGGGCTTGGGTAATAGCAGCCATTCTCGAGA |
| | ATGGCTGCTATTACCCAAGCTTTTTG |
| Hdac1 shRNA 2# | CCGGGCCAGTCATGTCCAAAGTAATCTCGAGA |
| | TTACTTTGGACATGACTGGCTTTTTG |
| Hdac1 shRNA 3# | CCGGGCGTTCTATTCGCCCAGATAACTCGAGTT |
| | ATCTGGGCGAATAGAACGCTTTTTG |
| Hdac2 shRNA 1# | CCGGCCCAATGAGTTGCCATATAATCTCGAGAT |
| | TATATGGCAACTCATTGGGTTTTTG |
| Hdac2 shRNA 2# | CCGGCGAGCATCAGACAAACGGATACTCGAGT |
| | ATCCGTTTGTCTGATGCTCGTTTTTG |
| Hdac2 shRNA 3# | CCGGCGATCAATAAGACCAGATAATCTCGAGA |
| | TTATCTGGTCTTATTGATCGTTTTTG |
| Dot1l shRNA 1# | CCGGGTCCAGTTTGTACTGTCAATACTCGAGTA |
| | TTGACAGTACAAACTGGACTTTTTG |
| Dot1l shRNA 2# | CCGGGCTGACCTACAATGACCTGATCTCGAGA |
| | TCAGGTCATTGTAGGTCAGCTTTTTG |
| Dot1l shRNA 3# | CCGGCCTCGGTTTACACAGCTTCAACTCGAGTT |
| | GAAGCTGTGTAAACCGAGGTTTTTG |
| control shRNA | non-target shRNA (SHC002, |
| | Smiga-Aldrich) |

TABLE 1-3
Gene sequences used in this study

| Gene | Sequence (5′ to 3′) |
|---|---|
| exo- | ATGGACTACAAAGACGATGACGACAAGGGATCCACTAGTAACG |
| DUX | GCCGCCAGTGTGCTGGAATTCTGGCTGAAGCTGGGTCTCCTGT |
| | GGGAGGCTCCGGAGTTGCCAGAGAGTCTAGAAGGAGGAGGAAG |
| | ACTGTGTGGCAAGCTTGGCAGGAGCAGGCCTTGTTGAGTACCT |
| | TCAAAAAGAAGAGATATCTCTCTTTTAAGGAAAGGAAGGAGTT |
| | GGCCAAAAGGATGGGGGTATCTGACTGCAGGATCAGAGTATGG |
| | TTCCAAAATAGGCGAAACAGATCAGGAGAGGAAGGGCATGCTT |
| | CAAAGAGGAGCATAAGAGGATCCAGGCGACTGGCCTCACCCCA |
| | GCTCCAGGAAGAATTGGGGAGTAGACCTCAGGGCAGAGGCATG |
| | AGATCCAGCGGGAGAAGGCCTAGAACTAGACTGACAAGTCTCC |
| | AGCTCAGGATCTTGGGGCAAGCATTTGAGAGAAATCCTAGACC |
| | AGGGTTTGCAACCAGGGAGGAGCTTGCTAGGGACACCGGCCTG |
| | CCAGAGGACACTATTCATATATGGTTTCAGAATAGAAGGGCTC |
| | GAAGAAGGCACAGGAGGGGAAGACCTACCGCCCAGGATCAGGA |
| | CCTCTTGGCTAGTCAAGGATCTGATGGTGCACCAGCCGGCCCA |
| | GAAGGAAGAGAAAGAGAGGGAGCTCAAGAAAATCTTCTTCCCC |
| | AAGAAGAGGCAGGAAGCACAGGAATGGACACATCCTCTCCATC |
| | TGATCTTCCTAGCTTTTGTGGTGAATCCCAACCCTTCCAAGTA |
| | GCACAACCAAGGGGAGCCGGACAACAAGAAGCACCTACAAGGG |
| | CAGGGAATGCTGGATCCCTCGAACCATTGCTGGACCAGTTGCT |
| | TGATGAGGTGCAAGTTGAGGAACCAGCACCTGCACCACTGAAC |
| | CTGGACGGAGATCCTGGTGGGAGAGTACATGAAGGGTCCCAAG |
| | AGAGCTTCTGGCCACAAGAAGAGGCAGGTTCAACTGGCATGGA |
| | TACCAGTTCTCCTTCTGATTCTAATTCTTTTTGTAGGGAGAGC |
| | CAACCAAGCCAGGTGGCCCAACCTTGTGGTGCTGGGCAGGAGG |
| | ATGCAAGGACTCAAGCTGATTCAACAGGCCCACTTGAACTGTT |
| | GTTGCTGGACCAACTGCTGGATGAAGTTCAGAAAGAAGAGCAT |
| | GTCCCAGTACCACTTGACTGGGGTAGAAATCCAGGAAGTAGAG |
| | AACACGAGGGCAGTCAAGACTCACTGCTTCCTCTGGAAGAGGC |
| | CGTTAATAGTGGGATGGATACAAGTATCCCATCAATATGGCCT |
| | ACTTTTTGTAGAGAGTCACAGCCACCACAGGTCGCACAACCAT |
| | CTGGGCCTGGACAGGCACAGGCACCAACTCAAGGAGGGAATAC |
| | TGACCCATTGGAGTTGTTCCTTTATCAACTTCTGGATGAAGTG |
| | CAGGTGGAGGAACATGCACCTGCTCCCCTCAACTGGGATGTGG |
| | ATCCTGGTGGAAGAGTGCATGAGGGGTCTTGGGAGAGCTTCTG |
| | GCCACAGGAAGAAGCAGGAAGTACTGGCCTTGATACCTCATCC |
| | CCTTCTGATTCCAATTCATTCTTTAGAGAGTCTAAACCTTCTC |
| | AAGTAGCCCAAAGGAGAGGTGCTGGCCAGGAGGATGCAAGGAC |
| | ACAGGCTGATTCTACCGGACCTCTGGAGCTGCTGCTCTTTGAC |
| | CAACTGCTGGACGAGGTGCAGAAAGAGGAACACGTCCCTGCCC |
| | CCCTTGACTGGGGAAGGAATCCCGGTAGCATGGAGCATGAAGG |
| | TTCCCAGGACAGCCTGCTCCCTCTGGAGGAAGCTGCTAATTCC |
| | GGCAGAGATACCTCAATACCTAGTATCTGGCCCGCTTTCTGCA |
| | GAAAGTCACAACCCCCACAAGTGGCACAGCCATCAGGACCTGG |
| | CCAGGCACAAGCACCTATACAAGGAGGTAATACAGATCCCCTG |
| | GAGTTGTTTCTGGATCAGCTTCTTACTGAAGTACAGTTGGAGG |
| | AGCAGGGCCCTGCTCCTGTGAACGTGGAAGAAACATGGGAGCA |
| | AATGGACACCACACCTGATTTGCCCTTGACCTCTGAGGAGTAC |
| | CAGACTCTTCTGGACATGTTGTGA |

In Vitro Induction of Trophoblast Stem-Like Cells from TPS Cells

TPS cells were digested into single cells and seeded onto feeder cells, which were cultured in TS condition medium: RPMI-1640 (Gibco, 11879-020) supplemented 309 with 20% ES qualified FBS, 1% L-Glutamine (Gibco, 25030-081), 1% sodium pyruvate (Gibco, 11360-070), heparin (1 ug/ml; Macklin, H811552-500KU), mouse FGF4 (25 ng/ml; Bioteche, 5846-F4), human bFGF (20 ng/ml; Novoprotein, C046). After 3-4 days, cells were passaged using TS medium. Flat TS-like colonies gradually emerged after passaging. Then the culture medium was changed into serum-free FAXY-TS medium: 50% Neurobasal, 50% DMEM/F12, 0.5% N2 supplement, 1% B27 supplement, 1% L-Glutamine, 1-thioglycerol (1.5×10−4 μM; Sigma, M6145), human bFGF (25 ng/ml; Novoprotein, C046), recombinant human activin A (20 ng/ml; Novoprotein, C687), XAV939 (10 μM; Selleck, S1180), and Y27632 (5 μM; Tocris, 1254).

In Vitro Induction of Primitive Endoderm-Like Cells from TPS Cells

TPS cells were differentiated to primitive endoderm-like cells over the course of 3 days by plating 2.5*10⁴ cells/cm² onto matrigel-coated plates in N2B27 medium supplemented with mouse FGF4 (50 ng/ml; Bioteche, 5846-F4), retinoic acid (10 nM; Sigma, R2625), 8-Bromo cAMP (1 mM; Selleck, S7857), CHIR-99021 (3 μM, Selleck, S1263). After the emergency of XEN-like cells, XEN cells were maintained in eXEN medium: RPMI-1640 supplemented 20% ES qualified FBS, 1% sodium pyruvate, 1% L-Glutamine, mouse FGF4(25 ng/ml; Bioteche, 5846-F4) and heparin (1 g/ml; Macklin, H811552-500KU).

### Immunofluorescence

The cells were fixed in 4% paraformaldehyde (DingGuo, AR-0211) at room temperature for 20 min and blocked with PBS (Corning, 21-040-CVR) that contained 0.2% Triton X-100 (Sigma-Aldrich, T8787) and 3% normal donkey serum (Jackson ImmunoResearch, 017-000-121) at room temperature for 1 h. The cells were incubated with primary antibodies at 4° C. overnight. Secondary antibodies (Jackson ImmunoResearch) were incubated at room temperature for 1 h after washing primary antibodies 3 times with PBS. The nuclei were stained with DAPI (Roche Life Science, 10236276001) at room temperature for 3 min and washed with PBS 3 times. The following primary antibodies were used: anti-ZSCAN4 (1:5000; MilliporeSigma, AB4340), anti-MuERVL-Gag (1:500; Epigentek, A-2801-100), anti-OCT4 (1:500; Abcam, ab181557), anti-OCT4 (1:200; Abcam, ab27985), anti-SOX2 (1:200; MilliporeSigama, AB5603), anti-EOMES (1:500; Abcam, ab183991), anti-TFAP2C (1:500; Abcam, ab218107), anti-PDGFRA (1:200; R&D, AF1062), anti-SOX17 (1:200; R&D, AF1924), anti-SOX7 (1:200; R&D, AF2766), anti-GATA6 (1:200; R&D, AF1700), anti-CDX2 (1:200; BioGenex, MU392A).

### Teratoma Assay

Mouse TPS cells were collected by trypsinization before injection. Approximately 10⁶ cells were injected sub-cutaneously into immunodeficient NPG mice by mixing with Matrigel. Teratomas generally developed within 2-6 weeks, and the animals were killed before the tumor size exceeded 1.5 cm in diameter. The teratomas were then digested into single cells using Collagenase IV. Then red blood cells were removed using Red Blood Cell (RBC) Lysis Buffer (Thermo Fisher Scientific, 00-4333-57). The digested teratoma cells were processed for 10× Genomics scRNA-seq.

### Chimeric Assay of Single-Cell Microinjection

Cells were digested by 0.05% trypsin-EDTA, and the digested cells were filtered through a 40 m cell strainer, centrifuged at 1,200-1,500 rpm for 3 min at room temperature. Supernatant were removed and cells were suspended using culture medium with the addition of Y-27632 (10 μM; Tocris, 1254), and placed on the ice before injection. After being placed on ice, the digested cells should be injected in 1 h, otherwise, another batch of cells were digested for the remaining injections.

Single cells were microinjected into 8-cell ICR diploid mouse embryos. The injected embryos were cultured in the culture medium with Y-27632 (10 μM; Tocris, 1254) for the first 4 h. For the generation of chimeric blastocysts, the embryos were transferred into the KSOM medium (Merck, MR-106-D). For the generation of single-cell derived in vivo chimeric conceptuses, chimeric embryos were cultured into KSOM medium with Y-27632 (10 μM; Tocris, 1254) addition in a humidified incubator under 5% CO2 at 37° C. overnight. Injected embryos were transferred to uterine horns of 0.5 post coitum pseudo-pregnant females. Fetal tissues, yolk sacs, placentas were dissected from conceptuses at E7.5, E10.5, E13.5 or E17.5 developmental stages.

### Chimeric Assay of Multiple-Cell Microinjection

For the generation of chimeric blastocyst, TPS cells were cultured in FAXY basal medium [50% Neurobasal, 50% DMEM/F12, 0.5% N2 supplement, 1% B27 supplement, 1% L-Glutamine, 1-thioglycerol (1.5×10−4 μM; Sigma, M6145), supplemented with human BMP4 (100 ng/ml; Stemimmune, HST-B4-0100) and bFGF (100 ng/ml; Novoprotein, C046) for 2 days. Cells were first digested by 0.05% trypsin-EDTA, and filtered through a 40 m strainer. Afterward, centrifuged at 1,200-15.00 rpm for 3 min. Cells were suspended using culture medium and placed on ice. 10-15 of the digested cells were microinjected into each 2/8-cell ICR or GFP diploid mouse embryo. Injected embryos were transferred into KSOM medium and cultured in a humidified incubator under 5% CO2 at 37° C. for 48-72 h till developed to E4.5, fixed and immunostaining, and more than 20 embryos were analyzed.

### Chimeric Assay of Single Blastomere of 8-Cell Embryo

Immunofluorescent 8-cell embryos' zona pellucida was removed by Acidic Tyrode's Solution (Sigma, T1788). When the zona pellucida disappeared, the 2-cell embryos were washed 3 times by M2 Medium (Sigma, M7167). Embryos were digested by TrypLE™ Select (Gibco, A1217702) 3 times, then DNase I (Gibco, 90083) twice. Blastomeres would separate after final digestion by TrypLE™ Select and DNase I in 37° C. for 5 min. Single immunofluorescent blastomere was aggregated with a zona pellucida removed wild type 8-cell embryo in AggreWell (Stemcell, 34415). Aggregated embryos were culture in KSOM in a humidified incubator under 5% CO2 at 37° C. overnight. Aggregated embryos were transferred to uterine horns of 0.5 post coitum pseudo-pregnant females. Fetal tissues, yolk sacs, placentas were dissected from conceptuses at E7.5 or E10.5developmental stages.

### Analysis of the Contribution of TPS Derivatives to E13.5 Fetal Gonads

TPS cells were microinjected into 8-cell ICR diploid mouse embryos. Injected embryos were transferred to uterine horns of 0.5 post coitum pseudo-pregnant females. E13.5 embryos were collected for analyzing the expression of tdTomato and OCT4-GFP.

### Immunofluorescence of E4.5 Embryo

E4.5 embryos were washed 3 times in PBS droplets, then fixed in 4% paraformaldehyde (DingGuo, AR-0211) droplets at room temperature for 20 min. PBS (Corning, 21-040-CVR) that contained 0.2% Triton X-100 (Sigma-Aldrich, T8787) and 3% normal donkey serum (Jackson ImmunoResearch, 017-000-121) were used to block embryos at room temperature for 1 h. Primary antibodies were diluted with blocking solution, then incubated embryos at 4° C. overnight. Embryos were washed 3 times in PBS droplets, then incubated with secondary antibodies (Jackson ImmunoResearch) at room temperature for 1 h. After final washes, blastocysts were transferred to confocal dish in PBS droplets covered with paraffin for imaging. Confocal microscope imaging was performed using Leica TCS-SP8. The following primary antibodies were used: anti-tdTomato (1:2000; SICGEN, AB8181-200), anti-OCT4 (1:200; Santa Cruz Biotechnology, sc-8626), anti-PDGFRA (1:200; R&D, AF1062), anti-CDX2 (1:200; BIOGENEX, MU392A), anti-EOMES (1:500; Abcam, ab183991), anti-TFAP2C (1:500; Abcam, ab218107), anti-CK8 (1:200; Abcam, ab53280).

### Immunofluorescence of E7.5 Embryo

E7.5 Embryos were Dissected in PBS, and then Embedded in O.C.T (SAKURA, 4583).

### Embryos

were frozen with liquid nitrogen vapor. Tissue sections of 10-15 mm thickness were cut in a cryostat microtome and transferred onto slides. Tissues were circled by PAP pen, then fixed in 4% paraformaldehyde (DingGuo, AR-0211) at room temperature for 15 min and blocked with PBS (Corning, 21-040-CVR) that contained 0.2% Triton X-100 (441 Sigma-Aldrich, T8787) and 3% normal donkey serum (Jackson ImmunoResearch, 017-000-121) at room temperature for 1 h. After blocking, placenta tissues were incubated with primary antibodies at 4° C. overnight. Secondary antibodies (Jackson ImmunoResearch) were incubated at room temperature for 1 h after washing primary antibodies 3 times with PBS. The nuclei were stained with DAPI (Roche Life Science, 10236276001) at room temperature for 3 mins and washed with PBS 3 times. Confocal microscope imaging was performed using Leica TCS-SP8 and Leica TCS-SP8 DIVE. The following primary antibodies were used: anti-tdTomato (1:2000; SICGEN, AB8181-200), anti-OCT4 (1:500; Abcam, ab181557), anti-EOMES (1:500; Abcam, ab183991), anti-SOX17 (1:200; R&D, AF1924).

### Immunofluorescence of E10.5 Placenta

E10.5 chimeric embryos were dissected in PBS. The fetus, yolk sac and placenta were separated using fine-pointed forceps. Placentas were embedded in O.C.T (SAKURA, 4583) and frozen with liquid nitrogen vapor. Tissues were circled by PAP pen, then fixed in 4% paraformaldehyde (DingGuo, AR-0211) at room temperature for 15 min and blocked with PBS (Corning, 21-040-CVR) that contained 0.2% Triton X-100 (Sigma-Aldrich, T8787) and 3% normal donkey serum (Jackson ImmunoResearch, 017-000-121) at room temperature for 1 h. After blocking, placenta tissues were incubated with primary antibodies at 4° C. overnight. Secondary antibodies (Jackson ImmunoResearch) were incubated at room temperature for 1 h. After washing primary antibodies 3 times with PBS. The nuclei were stained with DAPI (Roche Life Science, 10236276001) at room temperature for 3 mins and washed with PBS 3 times. Confocal microscope imaging was performed using Leica TCS-SP8 and Leica TCS-SP8 DIVE. The following primary antibodies were used: anti-tdTomato (1:2000; SICGEN, AB8181-200), anti-TFAP2C (1:500; Abcam, ab218107), anti-CK8 (1:500; Abcam, ab53280), anti-MCT4 (1:200; Millipore, AB3314P).

### Flow Cytometry Analysis of Chimeric Placental Tissues

Chimeric GFP positive placental tissues were gently isolated and digested into single cells using 0.1% Collagenase IV (Gibco, 17104019; dissolved in Ca2+/Mg2+ free PBS with 10% FBS and 100 g/ml DNase I). Suspensions were filtered through a cell strainer (100 mm). Then, the samples were analyzed on an Arial Sorp (BD Biosciences) or CytoFLEX(Beckman Coulter) to detect the presence of tdTomato and GFP expression. Data analysis was performed using FlowJo software (Ashland).

### Generation of Blastoid

TPS cells were dissociated into single cells by 0.05% trypsin-EDTA. Cells were suspended in induction medium comprising: 1:1:1 mixture of TS conditional medium, N2B27 basal medium and KSOM plus human BMP4 (100 ng/ml; Stemimmune, HST-B4-0100), human bFGF (100 ng/ml; Novoprotein, C046), mouse FGF4 (25 ng/ml; Bioteche, 5846-F4), LPA (10 μM; Sigma, 857228P) and Y27632 (10 μM; Tocris, 1254) on AggreWell (Stemcell, 34415). 3*10⁴ cells were seeded into one well. 4-6 days later, blastoids were formed.

### Immunofluorescence of Blastoids

Blastoids were washed 3 times in PBS droplets, then fixed in 4% paraformaldehyde (DingGuo,
AR-0211) droplets at room temperature for 20 min. PBS (Corning, 21-485 040-CVR) that contained 0.2% Triton X-100 (Sigma-Aldrich, T8787) and 3% normal donkey serum (Jackson ImmunoResearch, 017-000-121) were used to block embryos at room temperature for 1 h. Primary antibodies were diluted with blocking solution, then incubated embryos at 4° C. overnight. Embryos were washed 3 times in PBS droplets, then incubated with secondary antibodies (Jackson ImmunoResearch) at room temperature for 1 h. After final washes, blastoids were transferred to confocal dish in PBS droplets covered with paraffin for imaging. Confocal microscope imaging was performed using Leica TCS-SP8. The following primary antibodies were used: anti-OCT4 (1:500; Abcam, ab181557), anti-CDX2 (1:200; BioGenex, MU392A).

### Western Blot

For detecting histone proteins, the histone proteins were isolated from 5×10⁶ cells using Histone protein extract Kit (beibokit, BB-3117). For detecting 0-catenin protein, whole-cell protein extracts were isolated from 5*10⁶ cells using RIPA lysis buffer (Beyotime Technology Technology, P0013B) supplemented with protease inhibitor cocktail (Thermo Fisher Scientific, 78443) and phosphatase inhibitor cocktail (Thermo Fisher Scientific, 78428). The protein amount was determined using the Bicinchoninic acid (BCA) assay Kit (Applygen, P1511). Blots were incubated in 5% skimmed milk powder/TBST at room temperature for 1 hour, and then, they were incubated with the following antibodies in 5% BSA or 5% skimmed milk powder/TBST at 4° C. overnight: anti-histone H3 (1:1000, Abcam, ab1791), anti-acetyl-Histone H3 (1:1000, Millipore, 06-599), anti-acetyl-Histone H4(1:1000, Millipore, 06-866), anti-dimethyl-Histone H3 (Lys79) (1:1000, Millipore, 04-835), anti-β-catenin(1:2000, CELL SIGNALING, 8480), anti-GAPDH(1:4000, Applygen, C1312). The primary antibodies were washed using TBST, and the samples were further incubated with secondary antibodies for 1 hour at room temperature while shaking. The following secondary antibodies were used: goat anti-rabbit IgG, HRP-linked antibody (1:3,000; ZSGB-BIO, ZB-2301) and goat anti-mouse IgG, HRP-linked antibody (1:3,000; ZSGB-BIO, ZB-2305). The blots were developed using Western blotting luminol reagent (Santa Cruz, sc-2048).

Generation pf p53 and Dux Knockdown mEPS and mTPS Cell Lines

mEPS or mTPS cells carrying a Zscan4-Emerald GFP reporter were co-transfected with px330 plasmids (Addgene, 42230) encoding Cas9 and sgRNAs by nucleofection (4D-Nucleofector System, Lonza). The whole well cells were collected to extracted total RNA for further analyzed by Q-PCR.

shRNA Knockdown

Hdac1/2 or Dot1l shRNAs were transfected into EPS cells by nucleofection (4D-Nucleofector System, Lonza). Then EPS cells were cultured in LCDM for two days. On the third day, EPS cells were transfected with the same shRNAs by nucleofection, and the cells were seeded in the 4S condition or 4S condition without VPA/EPZ004777. The medium was changed daily. Puromycin (0.8ug/ml) was added in the culturing medium to enrich cells expressing shRNAs. After three days, the cells were collected and total RNAs were isolated for Q-PCR analysis.

### Induction of Oct4 Knockout in TPS Cells

Oct4 conditional knockout mouse ES cell line ZHBtc4 (Niwa, H., Miyazaki, J. & Smith, A. G. Quantitative expression of Oct-3/4 defines differentiation, dedifferentiation or self-renewal of ES cells. Nature genetics 24, 372-376, doi:10.1038/74199 (2000)) were converted into TPS cells using the 4S condition for more than 5 passages. Then tetracycline (1 g/ml) was added in the 4S condition to induce Oct4 knockout in ZHBtc4-TPS cells. After 3 days of treatment, the proliferation state of these cells was checked. Then treated and untreated cells were collected and total RNAs were isolated for Q-PCR analysis.

### Analysis of the Effect of 4S Chemical Cocktail on Totipotency Maintenance in Mouse Preimplantation Embryos

Mouse middle 2-cell embryos were collected and treated using the 4S chemical cocktail (VPA 500 μM, EPZ004777 1 μM, CD1530 0.5 μM, CHIR 99021 3 μM), which was added into the KSOM medium. The KSOM medium supplemented with DMSO was used as the control. 48 hours later, embryos were collected and lysed using Trizol reagent. Total RNAs were extracted from the lysates, which were further used for synthesizing cDNAs. cDNAs were prepared and amplified using the Smart-seq2 approach. The amplified cDNA product was diluted ten-fold as required by the qPCR template. Quantitative PCR analysis was conducted using the KAPA SYBR FAST qPCR Kit (KAPA Biosystems, KK4601) on a Bio RAD CFX Connect Real-Time System. The primers that were used for Q-PCR are listed in Table 1.

### Analysis of the Effects of HDAC Inhibition, DOTIL Inhibition, RAR Activation/Inhibition and LIF Signaling on Inducing Totipotency in TPS Cells Using Small Molecules

EPS cells were seeded onto feeder cells. To replace VPA, EPZ004777, CD1530 and CHIR 99021, these small molecules were removed from the 4S condition individually, and small molecules target the same target were added respectively. After 3 days of induction, cells were collected for further analysis. Small molecules replacing VPA included TSA (1 nM; Selleck, S1045), MS275 (1 μM; Selleck, S1053), Scriptaid (1 μM; Selleck, S8043), MGCD0103 (20 nM; Selleck, S1122). Small molecules replacing EPZ004777 included EPZ5676 (3 μM; Selleck, S5676) and SGC0946 (0.05 μM; Selleck, S7079). Small molecules replacing CD1530 included AM580 (0.05 μM; Selleck, S2933), ch55 (0.05 μM; MCE, HY-107397), Palovarotene (0.01 μM; MCE, HY-14799), CD3254 (50 nM; MCE, CD3254) and RA (0.5 M; MCE, HY-14649). Small molecules replacing CHIR 99021 included AZD2858 (2 μM; MCE, HY-15761), LY2090314(1 μM; MCE, HY-16294), BIO (2 μM; MCE, HY-10580), CHIR 98014 (2 μM; MCE, HY-13076), and SB216763 (10 μM; MCE, HY-216763). To inhibit RAR signaling during culturing TPS cells, RARγ inhibitor: LY2955303 (1 m, MCE, HY-107765); RARα/inhibitor: LE135 (2 m, MCE, HY-107436); RXR inhibitor: UV13003 (1 m, MCE, HY-107500) were individually added into the 4S medium. To inhibit LIF-Stat3 signaling during culturing TPS cells, AG490 (10 μM; MCE, HY-12000), Niclosamide (1 μM; MCE, HY-B0497), JAK inhibitor (10 μM; Millipore, 420097) were individually added into the 4S medium. After 4-6 passages, cells were collected for further analysis.

### Bulk RNA-Seq Analysis

Total RNA was isolated from mouse TPS and EPS cells using the RNeasy Mini Kit (Qiagen, 74106). RNA sequencing libraries were constructed by using magnetic beads with oligo (DT) enriched mRNA. After that, fragment buffer was added to break the mRNA into short segments. Using mRNA as template, a strand of cDNA was synthesized using six base random primers. Then buffer, dNTPs, DNA polymer I and RNase H were added to synthesize a two strand cDNAs, which were purified using AMPURE XP beads. The purified double stranded cDNAs were repaired, a-tailed and sequenced, and then the fragment size was selected by AMPURE XP beads. Finally, PCR amplification was carried out and PCR products were purified with AMPURE XP beads to obtain the final library. The fragmented and randomly primed 2×100-bp
paired-end libraries were sequenced using an Illumina HiSeq 2500. The generated sequencing reads were mapped against mouse genome build GRCm38.p6 for mouse using STAR v2.7.3a. The read counts for each gene were calculated, and the expression values of each gene were normalized using TPM. Hierarchical clustering analysis was performed using the PCA function from the package FactoMineR and the ward.D algorithm in R software. Differentially expressed gene analysis was performed using DESeq2 and filtered by adjusted P<0.05. Gene Ontology analysis was performed using the package topGO and org.Mm.eg.db. Single cell RNA sequencing data of mouse embryos from preimplantation (GSE45719 (Deng, Q. L., Ramskold, D., Reinius, B. & Sandberg, R. Single-Cell RNA-Seq Reveals Dynamic, Random Monoallelic Gene Expression in Mammalian Cells. Science 343, 193-196, doi:10.1126/science.1245316 (2014))) was reanalyzed and the original counts were normalized using TPM. The TPM matrix was used for calculating the average expressions of differently expressed genes between TPS and EPS cells in the preimplantation embryos at different developmental stages. Box plots were used to show the expression of these genes during preimplantation development. To identify totipotent signatures of 2-cell embryos, Seurat objects were constructed using the default parameters from the expression matrix of single cell RNA sequencing data of mouse preimplantation embryos (GSE45719). The top 2,000 most variable genes were identified using the ‘vst’ algorithm, which were further standardized and normalized for performing PCA analysis. Genes from the top 11 PCA components were used to construct the KNN map and UMAP map. The epiblast population was identified from the blastocyst cells by the co-expression of Oct4, Nanog and Sox2. Differently expressed genes between 2-cell embryos and epiblast cells were identified using FindMarkers (logfc.threshold=1, min.pct=0.6, test.use=‘DESeq2’). A total of 2,399 genes were identified as 2-cell embryo enriched genes (padj<0.0001).

### 10× Genomics Single Cell RNA Sequencing

Cells were washed and resuspended in 1×PBS (calcium and magnesium free) containing 10% FBS. Cell viability was determined by Count Star, and the density of living cells were adjusted to 300-600 living cells per microliter. Cell suspension was loaded onto the Chromium single cell controller (10× Genomics). Chromium Single Cell 3′ GEM, Library & Gel Bead Kit v3.1 (10× Genomics, 1000075) and Chromium Single Cell B Chip Kit (10× Genomics, 1000074) were used to generate single-cell gel beads in the emulsion according to the manufacturer's protocol. In short, single cells were suspended in PBS containing 0.04% BSA. About 6,000 cells were added to each channel, and the target cells were recovered (about 3,000 cells). Captured cells were lysed and the released RNA were barcoded through reverse transcription in individual GEMs. Reverse transcription was performed on a S1000TM Touch Thermal Cycler (Bio Rad) at 53° C. for 45 min, followed by 85° C. for 5 min, and hold at 4° C. The cDNA was generated and then amplified, and quality assessed using an Agilent 4200(performed by CapitalBio Technology, Beijing). Single-cell RNA-seq libraries were constructed using Single Cell 3′ Library and Gel Bead Kit V3.1 according to the manufacture's introduction. The libraries were finally sequenced using an IlluminaNovaseq6000 sequencer with a sequencing depth of at least 100,000 reads per cell with pair-end 150 bp (PE150) reading strategy (performed by CapitalBio Technology, Beijing).

### 10× Genomics ATAC Sequencing

Cells were washed and resuspended in 1×PBS (calcium and magnesium free) containing 10% FBS. Cell viability was determined by Count Star. The nuclei were isolated and washed according to the method provided by 10× Genomics: “Nuclei Isolation for Single Cell ATAC Sequencing (CG000169)”. Then the nucleus was resuspended by chilled Diluted Nuclei Buffer (10×Genomics; 2000153). The volume of Diluted Nuclei Buffer used to resuspend nuclei was based on the number of starting cells and the final target nuclei concentration. Countstar (Rigel S2) was used to count the nuclei. The nuclei were then immediately proceeded to construct single cell ATAC-seq libraries. The nuclei were partitioned into nanoliter-scale GEMs by using Chromium Chip E Single Cell Kit (Product Code 1000156) and Chromium Single Cell ATAC Library & Gel Bead Kit (Product Code 1000110). A pool of ˜750,000 10× Barcodes was sampled to separately and uniquely index the transposed DNA of each individual nucleus. Then the libraries were generated (performed by CapitalBio Technology, Beijing). The libraries were sequenced using an Illumina Novaseq sequencer with a sequencing depth of at least 25k read pairs per nucleus with pair-end 50 bp (PE50) reading strategy.

### Whole Genome Bisulfite Sequencing (WGBS)

Genomic DNAs were extracted using DNeasy Blood & Tissue Kits (QIAGEN, 69504). 100 ng genomic DNAs were fragmented into around 200 bp by Covaris. Then DNAs were bisulfite-converted using EZ DNA Methylation-Gold™ Kit (Zymo, D5005). Bisulfite-converted DNA was captured using Accel-NGS Methyl-Seq DNA Library Kit (Swift Biosciences, 30024). Library samples were subjected to Illumina Nova-seq 6000 sequencing system.

### Single-Cell RNA-Seq Preprocess

The single-cell RNA-seq data were collected and mapped to mouse reference genome mm10 using Cell Ranger 3.1.0 for all our samples. We performed preprocess using Seurat 3.2.3 (Stuart, T. et al. Comprehensive Integration of Single-Cell Data. Cell 177, 1888-1902 e1821, doi:10.1016/j.cell.2019.05.031 (2019)) in R environment version 3.6.3. In detail, quality control was firstly performed to remove the cells with (i) total UMI counts less than 2000, (ii) detected gene number less than 1500, or (iii) mitochondrial UMI counts more than 20%. The normalization was performed using function NormalizeData with default parameters. We performed the standard Seurat clustering pipeline using the following functions: FindVariableFeatures with 2000 genes, ScaleData, RunPCA, FindNeighbors with first 10 PCs, and FindClusters with resolution 1.2. Then we checked the doublet scores using function doubletCells of R package scran for each sample. We found the scores evenly distributed across clusters, indicating there is no inter-cluster doublet detected, so we kept all cells for later analysis. We performed batch effect correction using R package Harmony (Korsunsky, I. et al. Fast, sensitive and accurate integration of single-cell data with Harmony. Nat Methods 16, 1289-1296, doi:10.1038/s41592-019-0619-0 (2019)). The Uniform Manifold Approximation and Projection (UMAP) dimension reduction was performed with first 10 corrected PCs using function RunUMAP. The differentially expressed genes (DEG) were analyzed using function FindMarkers based on normalized gene expression.

### Transcriptional Integration Analysis of Embryo Development States and Different Stem Cells

### Public Datasets Collection

We collected 12 public datasets to construct a mouse embryonic development trajectory as reference (GSE45719 from Deng et al., 2014 (Deng, Q. L., Ramskold, D., Reinius, B. & Sandberg, R. Single-Cell RNA-Seq Reveals Dynamic, Random Monoallelic Gene Expression in Mammalian Cells. Science 343, 193-196, doi:10.1126/science.1245316 (2014)); GSE109071 from Cheng et al., 2019 (Cheng, S. L. et al. Single-Cell RNA-Seq Reveals Cellular Heterogeneity of Pluripotency Transition and X Chromosome Dynamics during Early Mouse Development. Cell Reports 26, 2593-+, doi:10.1016/j.celrep.2019.02.031 (2019)); GSE44183 from Xue et al., 2013 (Xue, Z. G. et al. Genetic programs in human and mouse early embryos revealed by single-cell RNA sequencing. Nature 500, 593-+, doi:10.1038/nature12364 (2013)); GSE71434 from Zhang et al., 2016 (Zhang, B. J. et al. Allelic reprogramming of the histone modification H3K4me3 in early mammalian development. Nature 537, 553-+, doi:10.1038/nature19361 (2016)); GSE66582 from Wu et al., 2016 (Wu, J. Y. et al. The landscape of accessible chromatin in mammalian preimplantation embryos. Nature 534, 652-+, doi:10.1038/naturel8606 (2016)); GSE70605 from Liu et al., 2016 (Liu, X. Y. et al. Distinct features of H3K4me3 and H3K27me3 chromatin domains in pre-implantation embryos. Nature 537, 558-+, doi:10.1038/nature19362 (2016)); GSE100597 from Mohammed et al., 2017 (Mohammed, H. et al. Single-Cell Landscape of Transcriptional Heterogeneity and Cell Fate Decisions during Mouse Early Gastrulation. Cell Reports 20, 1215-1228, doi:10.1016/j.celrep.2017.07.009 (2017)); GSE84892 from Posfai et al., 2017 (Posfai, E. et al. Position- and Hippo signaling-dependent plasticity during lineage segregation in the early mouse embryo. eLife 6, 24, doi:10.7554/eLife.22906 (2017)); DRP005519 from Israel et al., 2019 (Israel, S. et al. An integrated genome-wide multi-omics analysis of gene expression dynamics in the preimplantation mouse embryo. Sci Rep 9, 13356, doi:10.1038/s41598-019-49817-3 (2019)); GSE121708 from Argelaguet et al., 2019 (Argelaguet, R. et al. Multi-omics profiling of mouse gastrulation at single-cell resolution. Nature 576, 487-+, doi:10.1038/s41586-019-1825-8 (2019)); GSE136714 from Wang et al., 2021 (Wang, Y. et al.

Single-cell multiomics sequencing reveals the functional regulatory landscape of early embryos. Nat. Commun. 12, 14, doi:10.1038/s41467-021-21409-8 (2021)); GSE145609 from Posfai et al., 2021 (Posfai, E. et al. Evaluating totipotency using criteria of increasing stringency. Nat. Cell Biol. 23, 32, doi:10.1038/s41556-020-00609-2 (2021))). Form these datasets, we used cells from zygote to E7.5 EPI, together with extraembryonic lineages. The cells of epiblast lineage after embryonic day 5 were defined as post-implantation epiblasts.

We also collected 4 stem cell datasets for comparison with our data (GSE33923 from Macfarlan et al., 2012 (Macfarlan, T. S. et al. Embryonic stem cell potency fluctuates with endogenous retrovirus activity. Nature 487, 57-+, doi:10.1038/naturell244 (2012)); GSE168728 from Shen et al., 2021 (Shen, H. et al. Mouse totipotent stem cells captured and maintained through spliceosomal repression. Cell 184, 2843-+, doi:10.1016/j.cell.2021.04.020 (2021)); GSE74155 from Chen et al., 2016 (Chen, G. et al. Single-cell analyses of X Chromosome inactivation dynamics and pluripotency during differentiation. Genome Res. 26, 1342-1354, doi:10.1101/gr.201954.115 (2016)); GSE145609 from Posfai et al., 2021 (Posfai, E. et al. Evaluating totipotency using criteria of increasing stringency. Nat. Cell Biol. 23, 32, doi:10.1038/s41556-020-00609-2 (2021))). From these datasets, ESCs, EPSCs, Primed EpiSCs, 2CLCs, TBLCs were used in later analyses.

Raw sequencing data of all public datasets were downloaded and performed preprocess following the same procedures to get normalized expression data using the same measurement with our data (logTP10K, log normalized transcripts per 10 thousand).

### Embryonic Development Trajectory Construction

To integrate multiple datasets together to form a continuous development trajectory, we performed latent semantic indexing (LSI) projection as previously described (Granja, J. M. et al. Single-cell multiomic analysis identifies regulatory programs in mixed-phenotype acute leukemia. Nat Biotechnol 37, 1458-1465, doi:10.1038/s41587-019-0332-7 (2019)). In detail, we used GSE45719 and GSE109071 as ‘stem’ datasets and combined them directly. We chose these two datasets for the reason (i) they were both sequenced using Smart-Seq2, (ii) they both had enough cell number for each development state to identify biological variance along the trajectory, and (iii) they were widely used for comparison in previous researches. We performed LSI dimension reduction to cells of these two datasets with top 2000 variable genes. Other 10 datasets were then projected to the LSI space. The coordinates of all cells in LSI space were only used to perform visualization.

### Stem Cell Data Integration

To compare stem cells from multiple datasets with embryonic development stages, we projected the stem cell data to the LSI space mentioned above. Due to the low sequencing depth of 10× platform for each cell, we combined the cells in our data and GSE168728 to pseudo-bulk samples. The first two LSI dimension of stem cell samples and cells of epiblast lineage were used to visualize. The cells of the same development stage were represented as one point at the median of their coordinates.

### Calculation of Embryonic Development Stage Signatures

We calculated gene signature for each embryonic development stage by combing multiple DEG fold change values to a comprehensive score for each gene using the entropy weight method (EWM). The DEGs were calculated using DESeq2 (Love, M. I., Huber, W. & Anders, S. Moderated estimation of fold change and dispersion for RNA-seq data with DESeq2. Genome Biol 15, 550, doi:10.1186/s13059-014-0550-8 (2014)) for multiple datasets, respectively.

### Single-Sample Gene Set Enrichment Analysis

We performed single-sample Gene Set Enrichment Analysis (ssGSEA) to quantify the similarity between stem cell samples and embryonic development stages using R package GSVA. As control, cells along the embryonic development trajectory were combined and performed the same analysis. The enrichment score for each signature was further normalized by dividing the value that positive control can reach for better visualization.

### Embryonic Stage Identity Analysis

To evaluate the degree of similarity between stem cell samples and given embryonic stages, we performed a quadratic programming-based deconvolution analysis using the R package quadprog as previously described (Treutlein, B. et al. Dissecting direct reprogramming from fibroblast to neuron using single-cell RNA-seq. Nature 534, 391-395, doi:10.1038/naturel8323 (2016)).

### Transcriptional Integration Analysis of Blastocysts and Blastoids

We constructed a blastocyst development trajectory using the cells after E3.5 subset from the whole embryonic development trajectory in the same procedure as mentioned above. Our blastoid data were collected and preprocessed the same as described above. Two public datasets were collected for comparison (GSE99786 from Rivron et al., 2018 (Rivron, N. C. et al. Blastocyst-like structures generated solely from stem cells. Nature 557, 106-+, doi:10.1038/s41586-018-0051-0 (2018)); GSE134240 from Sozen et al., 2019 (Sozen, B. et al. Self-Organization of Mouse Stem Cells into an Extended Potential Blastoid. Dev. Cell 51, 698-+, doi:10.1016/j.devcel.2019.11.014 (2019))). The LSI projection, ssGSEA, and identity score analysis was performed as described above.

### Single-Cell Gene Regulatory Network Analysis

We performed Single-Cell rEgulatory Network Inference and Clustering (SCENIC) (Aibar, S. et al. SCENIC: single-cell regulatory network inference and clustering. Nat Methods 14, 1083-1086, doi:10.1038/nmeth.4463 (2017)) analysis to our data following the standard pySCENIC pipeline (https://pyscenic.readthedocs.io/en/latest/index.html). The regulon activities for cells in other datasets were calculated using R package AUCell.

### Single-Cell ATAC-Seq Analysis

The single-cell ATAC-seq data were collected and mapped to mouse reference genome mm10 using Cell Ranger ATAC 1.2.0 for all our samples. The downstream analyses were performed following the standard ArchR (Granja, J. M. et al. ArchR is a scalable software package for integrative single-cell chromatin accessibility analysis. Nat Genet 53, 403-411, doi:10.1038/s41588-021-00790-6 (2021)) pipeline (https://www.archrproject.com).

We collected public ATAC-seq data of mouse preimplantation embryos (GSE66581 from Wu et al., 2016) for comparison analysis.

### WGBS Analysis

We followed the standard Bismark (Krueger, F. & Andrews, S. R. Bismark: a flexible aligner and methylation caller for Bisulfite-Seq applications. Bioinformatics 27, 1571-1572, doi:10.1093/bioinformatics/btr167 (2011)) workflow to perform mapping, deduplication, and methylation extraction (https://github.com/FelixKrueger/Bismark) for all our samples. Only CpG sites were used for further analysis and visualization.

We collected public methylome data of mouse embryos (GSE56697 from Wang et al., 2014 (Wang, L. et al. Programming and Inheritance of Parental DNA Methylomes in Mammals. Cell 157, 979-991, doi:10.1016/j.cell.2014.04.017 (2014))) for comparison analysis.

### Data Availability

The RNA-seq, WGBS and scRNA-seq, scATAC-seq data generated during this study are available at GEO: GSE183522.

### Results and Discussion

Identification of a Chemical Cocktail that Establish Totipotent-Like Stem Cells In Vitro

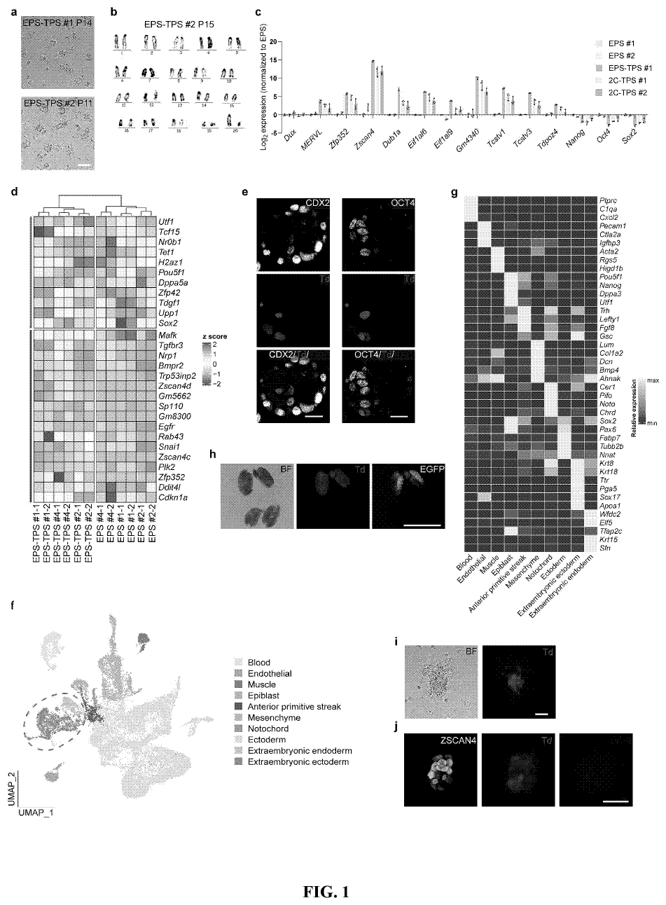

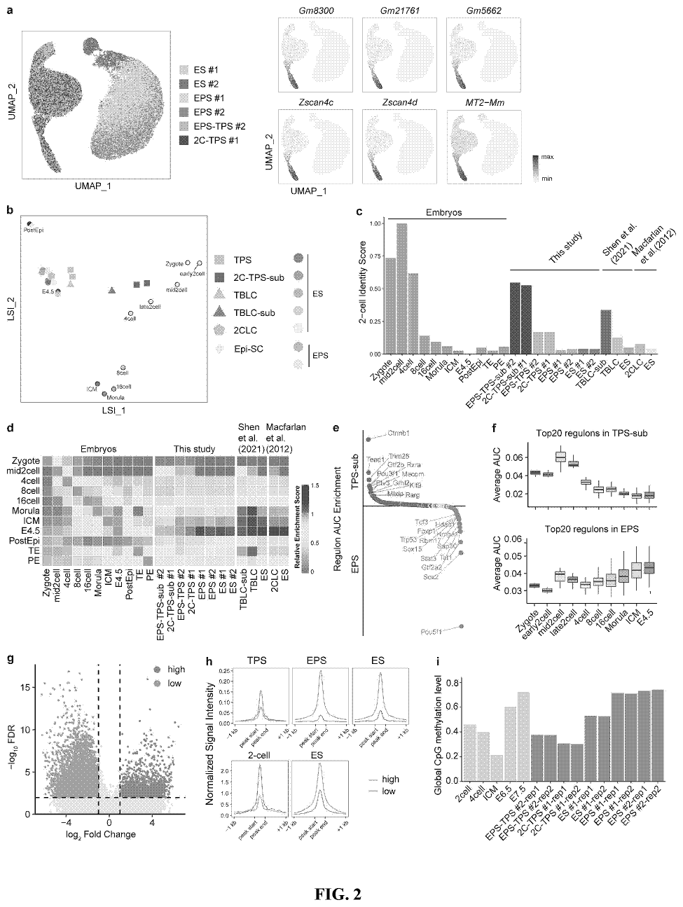

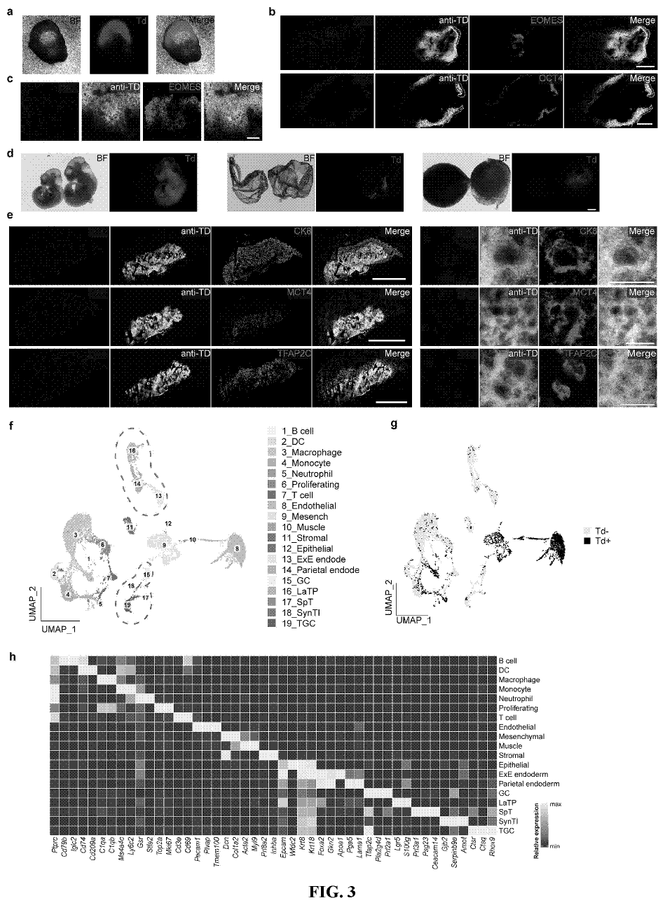

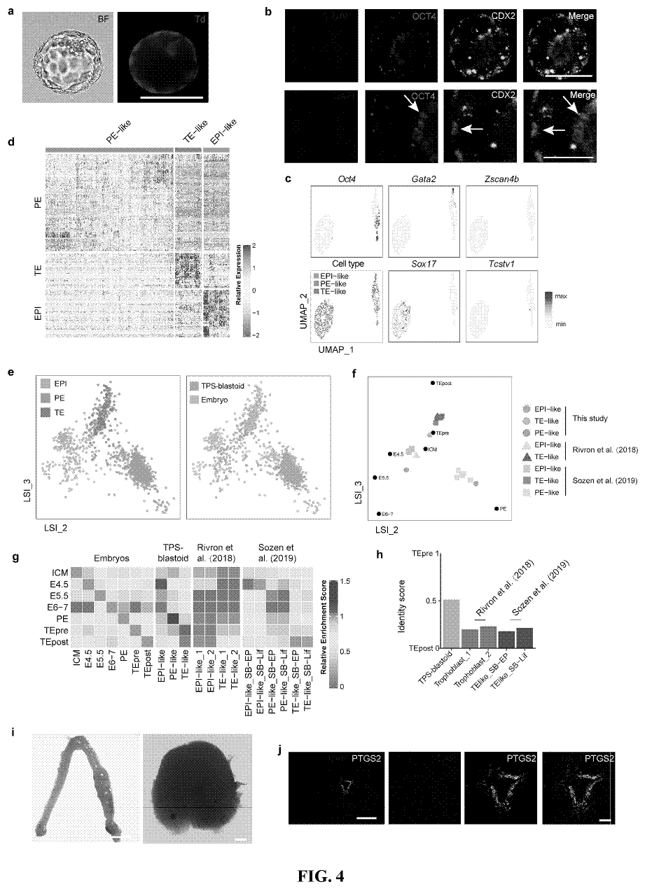

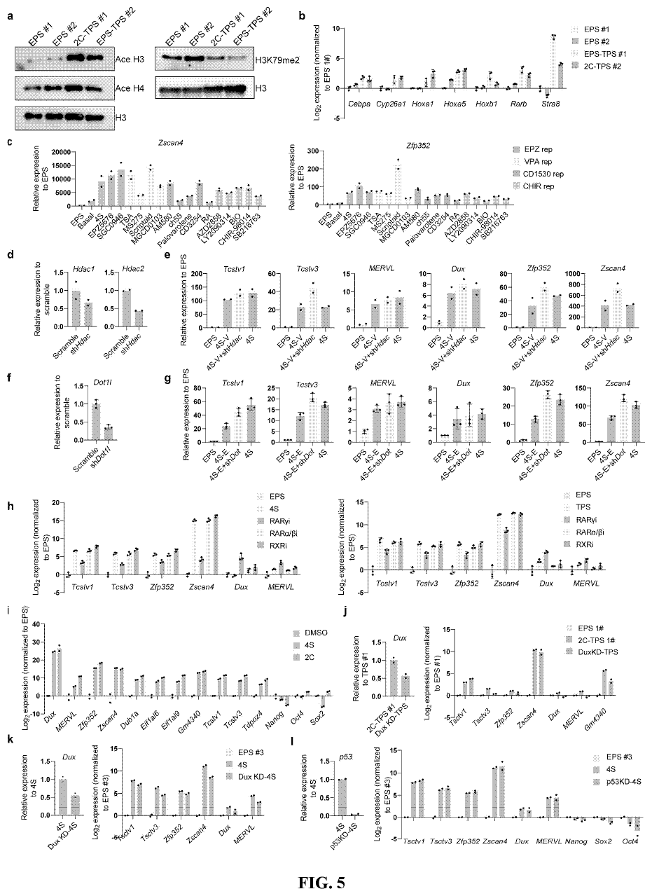

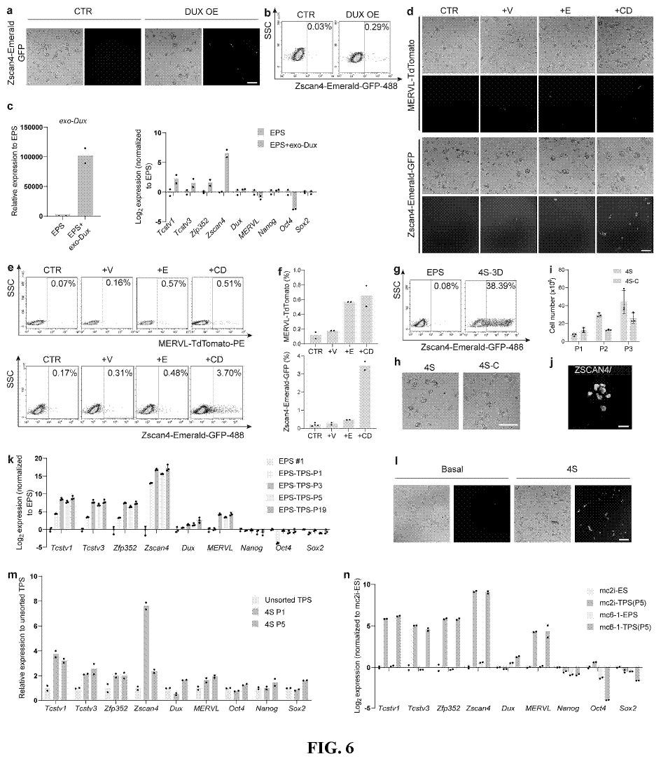

To identify small molecules that can induce totipotent features in mouse EPS cells, we used cell lines carrying a MERVL-TdTomato or a Zscan4-Emerald GFP reporter. As the positive control, Dux overexpression was performed in these reporter cells (FIG. 6a-c). N2B27 medium was used for screening and Wnt signaling agonist CHIR 99021 was also added for promoting cell proliferation. In the primary screen, we focused on identifying small molecules that can increase the percentage of Zscan4-Emerald GFP+ cells, and identified more than 50 primary hits. These candidates were further evaluated using MERVL-tdTomato reporter cells and small molecules that can also induce MERVL-tdTomato were selected out for further determining their optimal dosage in activating Zscan4-Emerald and MERVL-tdTomato expression without inducing significant toxicity. After the titration, we found that CD1530 was most effective in inducing Zscan4-GFP and MERVL-tdTomato expression (FIG. 6d-f). Therefore, we tested the effect of combing CD1530 with other selected candidates in inducing totipotent marker genes, and found that VPA and EPZ004777 showed a synergistic effect with CD1530 in inducing totipotent markers (FIG. 6g). We also tested whether CHIR 99021 could be omitted from the cocktail, and found that cell proliferation was greatly reduced under such condition during the conversion (FIG. 6h-i). Therefore, CHIR 99021 was included in the chemical cocktail.

We further analyzed whether cells cultured under the 4 small molecules (4S) condition could be stably maintained in vitro as well as preserve the totipotent features. 4S-cultured cells can be maintained and passaged for more than 15 passages with normal karyotype (FIG. 1a-b). After long-term culture (more than 10 passages), 4S-treated cells still maintained the expression of totipotent markers such as Zscan4, Zfp352, Tcstv1, Tcstv3, MERVL element, Dux, Dubla, Eif1al6, Ei1al9, Gm4340 and Tdpoz4 (FIG. 1c), and the protein expression of ZSCAN4 could be detected in these cells (FIG. 6j). Meanwhile, pluripotency markers, including Oct4, Nanog and Sox2, were significantly downregulated in these cells (FIG. 1c). We also compared the expression of totipotent marker genes in 4S-treated cells at different passages, and found that cells after passage 5 showed a relatively stable upregulation of totipotent marker genes, implying at least 5 passages are required for obtaining stable totipotent features in the 4S-treated cells (FIG. 6k). Consistent with Q-PCR analysis, bulk RNA-sequencing analysis also confirmed the upregulation of multiple totipotent marker genes as well as downregulation of multiple pluripotent marker genes (FIG. 1d).

To analyze the stability of cells expressing totipotent marker genes, we also enriched these cells using a Zscan4-Emerald GFP reporter and cultured them for 5 passages in vitro. 4S-treated cells proliferated well after the sorting, whereas the purified cells cultured without the 4 small molecules grew poorly (FIG. 6l). Further Q-PCR analysis showed that the expression of totipotent marker genes could be maintained in the 4S-treated cells after purification and culturing (FIG. 6m). We also tested whether the 4S condition could induce totipotent marker gene expression in mouse ES cells, and found that 4S-treated ES cells upregulated multiple totipotent marker genes after 5 passages (FIG. 6n). Collectively, these results indicated that 4S-treated cells acquired totipotent molecular features and were distinct from conventional pluripotent cell types.

Because the 4S-treated cells exhibited totipotent molecular features, we further explored whether they possessed extraembryonic developmental potentials in vitro. To this end, 4S-treated cells were cultured and passaged in a mouse trophoblast stem (TS) cell culture medium. After 2-4 passages, multiple flat mouse TS-like colonies emerged, which could be further passaged in mouse TS medium. Immunofluorescent analysis showed that these TS-like cells expressed TS markers including EOMES, TFAP2C CDX2, and SOX2 (FIG. 7a), suggesting they acquired they identity of trophoblast lineages. Consistent with these results, 4S-treated cells could also be induced into cells expressing markers of primitive endoderm (PE) upon culturing in the medium that induce PE differentiation (FIG. 7b). Next, we evaluated the developmental potentials of 4S-treated cells by performing in vitro chimeric experiments. 4S-treated cells with fluorescent reporter were injected into 8-cell mouse embryos which were cultured in vitro for 48 hours. Immunofluorescent analysis of the chimeric mouse embryos showed that derivatives from 4S-treated cells expressed markers of trophectoderm (TFAP2C, CK8, CDX2, EOMES), epiblast (OCT4) and primitive endoderm (PDGFRα) (FIG. 1e and FIG. 8).

To further explore the developmental potentials of 4S-treated cells in vivo, we injected these cells in immune-deficient mice and obtained teratomas, which were analyzed using single cell RNA sequencing analysis. Notably, in addition to the presence of multiple embryonic lineages in the teratomas, we found that teratomas derived from 4S-treated cells also contained extraembryonic lineages (FIG. 1f-g). Next, we examined the in vivo chimeric ability of 4S-treated cells, and found that 4S-treated cells after 5 passages showed relatively higher chimeric ability (Table 2). We also tested the germline competence of 4S-treated cells by injecting them into 8-cell embryos and transplanting the injected embryos in vivo to generate chimeric embryos. Analysis of the E13.5 chimeric embryos showed that derivatives of these cells can efficiently form chimerism in the fetal gonads, which expressed OCT4-GFP (FIG. 1h). Collectively, these results suggest that cells cultured under this new condition could be maintained long-term while preserving the ability to express totipotency marker genes and bi-developmental potentials, therefore we designated these cells as totipotent potential stem cells (TPS cells).

TABLE 2
Chimeric summary
Summary of 8-cell embryo chimeric assay by injecting TPS
cells from different passages

| | | | | injected cell |
|---|---|---|---|---|
| | | | Contributed | number |
| Detected | | Recovered | into both | (cells/ |
| stage | Passage | embryos | emb&extraemb | number) |
| 5.5 | P3 | 11 | 1 | 10~15 |
| 5.5 | P4 | 7 | 1 | 10~15 |
| 7.5 | P4 | 11 | 0 | 1 |
| 10.5 | P5 | 10 | 3 | 1 |
| 10.5 | P6 | 9 | 2 | 1 |
| 10.5 | P8 | 20 | 8 | 1 |

Summary of chimeric assay by injection of a single cell into
8-cell embryo derived from E7.5

| | Injected | Recovered | Contributed into both |
|---|---|---|---|
| Cell lines | embryos | embryos | emb&extraemb |
| TPS 1# | 44 | 26 | 6 |
| TPS 2# | 49 | 18 | 6 |
| TPS 3# | 48 | 15 | 3 |

Summary of chimeric assay by injection of a single cell into
8-cell embryo derived from E10.5

| | Injected | Recovered | Contributed into both |
|---|---|---|---|
| Cell lines | embryos | embryos | emb&extraemb |
| TPS 1# | 65 | 30 | 11 |
| TPS 2# | 66 | 22 | 12 |

Summary of chimeric assay by injection of a single
cell into 8-cell embryo without CHIR addition

| | Recovered | Contributed into both |
|---|---|---|
| Cell lines | embryos | emb&extraemb |
| 4S-C #1 | 93 | 3 |
| 4S-C #2 | 62 | 1 |

We further explored whether TPS cells can be directly derived from 2-cell embryos. To this end, 2-cell mouse embryos without zonal pellucida were seeded on feeder cells and cultured under the 4S condition. We found that some embryos transformed into compact cell clumps under the 4S condition followed by the emergence of outgrowth (FIG. 1i). Notably, immunofluorescent analysis revealed that totipotent marker ZSCAN4 could be detected in the outgrowth whereas the expression of pluripotent marker OCT4 was greatly reduced when compared with that in the 2i/LIF treated outgrowth (FIG. 9). The outgrowth can be further passaged, resulting in the subsequent emergence of domed TPS colonies, which showed expression of ZSCAN4 (FIG. 1j). Similar to TPS cells that were induced from EPS cells, TPS cells established from 2-cell embryos also expressed totipotent marker genes (FIG. 1c). These results indicate that the 4S condition enables the derivation of TPS cells directly from 2-cell mouse embryos.

TPS Cells Share Transcriptomic Features with 2-Cell Blastomeres

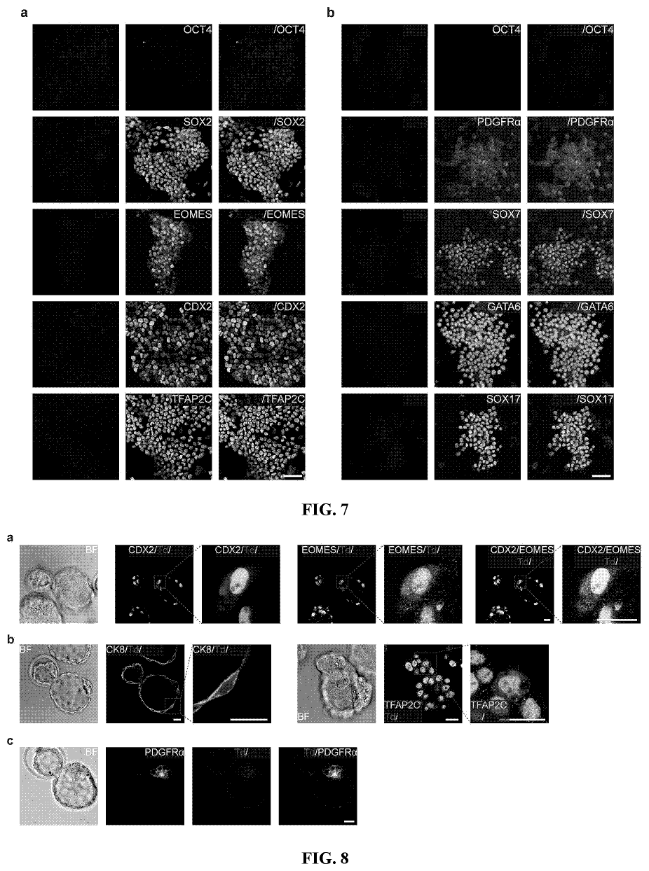

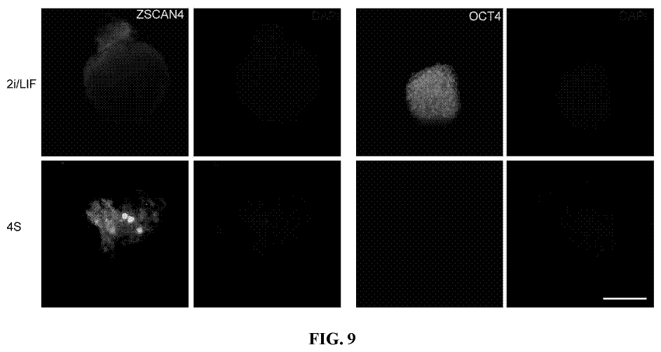

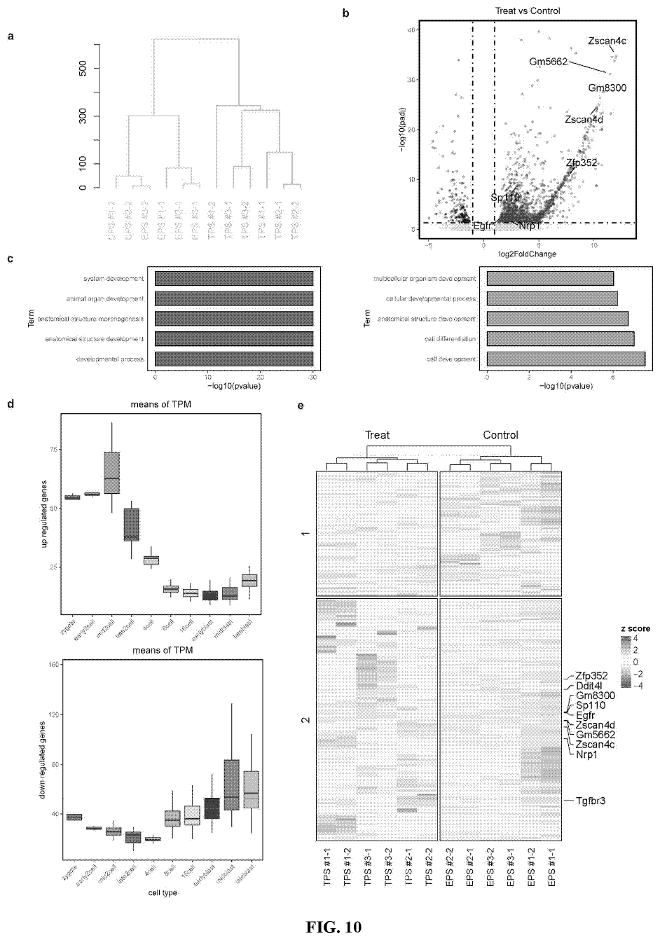

To explore the molecular features of TPS cells, we analyzed the transcriptomic differences between TPS cells and EPS cells using bulk-RNA sequencing data. Hierarchical clustering analysis showed that the global transcriptome of TPS cells was distinct from that of EPS cells (FIG. 10a). Identification of genes that were differently expressed between TPS cells and EPS cells showed the presence of representative totipotent marker genes, such as Gm5662, Zscan4c and Zfp352 (FIG. 10b). We further performed Gene Ontology (GO) analysis and found that the differently expressed genes are majorly involved in the regulation of development (FIG. 10c).

Next, we analyzed the expression levels of these differentially expressed genes in early preimplantation development. Notably, genes upregulated in TPS cells were also highly expressed at the 2C-embryo stage, the expression of which was gradually decreased at the blastocyst stage (FIG. 10d). On the contrary, genes downregulated in TPS cells were more abundantly expressed at the blastocyst stage but not the 2C-embryo stage (FIG. 10d). We also identified totipotent signatures including 2,399 genes that are upregulated in 2-cell totipotent embryos in comparison with epiblast cells from blastocysts using published single cell RNA sequencing data of preimplantation mouse embryos (Deng, Q., Ramsköld, D., Reinius, B. & Sandberg, R. Single-cell RNA-seq reveals dynamic, random monoallelic gene expression in mammalian cells. Science 343, 193-196, doi:10.1126/science.1245316 (2014)). Among these genes, TPS cells significantly upregulated 1,585 genes when compared with EPS cells (FIG. 10e). These data suggest that TPS cells possess transcriptomic features that are specific to 2-cell embryos.

To further explore the transcriptomic features of TPS cells, we performed single cell RNA sequencing using TPS cells. As the controls, the single cell RNA sequencing data of mouse EPS cells, ES cells, 2C-like cells and recently reported totipotent blastomere-like cells (TBLCs) were also analyzed. In consistent with bulk RNA-sequencing analysis, we found that the majority of TPS cells expressed multiple totipotent marker genes (FIG. 2a), the expression level of which varied among the population. Notably, the varied expression of totipotent marker genes was also observed in TBLCs (FIG. 11a), which is consistent with one recent report (Lin, P. Y. et al. Comparative Analyses of Single-Cell Transcriptomic Profiles between In Vitro Totipotent Blastomere-like Cells and In Vivo Early Mouse Embryonic Cells. Cells 10, doi:10.3390/cells10113111 (2021)). We further compared the transcriptome of these in vitro cell types to that of early embryos from 2-cell to E7.5 stage (FIG. 2b), and applied an analytical technique based on quadratic programming to quantify the transcriptomic similarity of these cell types to 2-cell embryos (Treutlein, B. et al. Dissecting direct reprogramming from fibroblast to neuron using single-cell RNA-seq. Nature 534, 391-395, doi:10.1038/nature18323 (2016)). The identity score of TPS cells was higher than that of other cell types (FIG. 2c), suggesting that TPS cells transcriptomically more resemble 2-cell embryos when compared to other cell types.

Because the expression of totipotent marker genes varied among the TPS cell population, we further explored the subpopulations that reside in the TPS cell population, and identified a portion of cells (average 9.14%) in the TPS cell population that transcriptomically resembled middle-to-late 2-cell embryos (FIG. 2b). Importantly, the identity score of this TPS subpopulation (TPS 2C-subpopulation) is the highest among all the analyzed in vitro cell types and subpopulations (FIG. 2c), which was also supported by single sample gene set enrichment analysis (ssGSEA) (FIG. 2d). We also analyzed the transcriptomic difference between the TPS 2C-subpopulation and 2-cell embryos. GO analysis revealed that genes differently expressed between this TPS 2C-subpopulation and 2-cell embryos are majorly involved in the regulation of mitotic cell cycle, proteasomal protein catabolism, nucleoside triphosphate metabolism, and oxidative phosphorylation (data not shown). Using SCENIC, we further analyzed the regulatory network of the TPS 2C-subpopulation and compared it with that of EPS cells. Notably, enrichment of totipotent regulators was upregulated in this population, whereas that of pluripotent regulators was downregulated (FIG. 2e-f). Collectively, these data suggest that TPS cells shared totipotent transcriptional features with 2-cell embryos and a TPS cell subpopulation transcriptomically resemble middle-to-late 2C embryos.

TPS Cells Share Epigenetic Features with 2-Cell Blastomeres

To characterize the epigenetic features of TPS cells, we first performed the assay for transposase-accessible chromatin using sequencing (ATAC-seq) on these cells at the single cell level. As the control, EPS cells and ES cells were also analyzed. Compared to EPS and ES cells, we identified 1,857 open and 8,903 closed peaks that were uniquely enriched at annotated or putative enhancers and promoters in TPS cells (FIG. 2g). The unique opened loci in TPS cells include multiple genes that are specifically expressed in 2-cell embryos, such as Zfp352, Zscan4c and Tcstv1 (FIG. 11b). We further analyzed the distribution of TPS-specific open and closed peaks in 2-cell embryos and mouse ES cells. Notably, TPS-specific opened loci were also highly opened in 2-cell embryos, whereas TPS-specific closed loci were in a more closed state in 2-cell embryos (FIG. 2h). We further analyzed the global DNA methylation profiles of TPS, EPS and ES cells using whole-genome bisulfite sequencing (WGBS). The global DNA methylation level in TPS cells was greatly reduced when compared to that in EPS and ES cells (FIG. 2i), which was similar to that in 2-cell embryos. In addition, we also observed that the levels of DNA methylation were reduced in the loci of representative totipotent genes, such as Zfp352, Zscan4d, and Tcstv1 (FIG. 11c). Taken together, these results suggest that TPS cells share epigenetic features with 2-cell blastomeres in the aspects of accessible chromatin landscape and DNA methylation profiles.

### TPS Cells can Generate Both Embryonic and Extraembryonic Lineages In Vivo

To rigorously analyze the developmental potentials of TPS cells, we first tested their ability of generating embryonic and extraembryonic lineages in E7.5 mouse embryos in vivo. To this end, single tdTomato reporter-labeled TPS cells were injected into 8-cell embryos that were transferred in vivo. As the control, single 8-cell blastomeres were also injected (FIG. 12a). Notably, we observed the contribution of single TPS derivative cells into both embryonic and extraembryonic regions in E7.5 mouse chimeric embryos (FIG. 3a and Table 2). Immunofluorescent analysis further confirmed that extraembryonic chimeric cells expressed EOMES in the region of extraembryonic ectoderm (ExE) (FIG. 3b-c), suggesting that they adopted an extraembryonic trophoblast fate. Meanwhile, we also noticed that cells that integrated into the visceral endoderm (VE) region expressed VE markers SOX17 (FIG. 12b), suggesting their VE identity.

Next, we analyzed the developmental potentials of single TPS cells in E10.5 mouse conceptuses (Table 2). The injected mouse embryos were transferred in vivo and recovered at E10.5 stage. Notably, similar to blastomere of 8-cell embryos (FIG. 12c), wide chimeric contribution of single TPS derivative cells to embryo, placenta and yolk sac was observed (FIG. 3d), which was also confirmed by FACS analysis (FIG. 12d). To further confirm the trophoblast identity of chimeric cells in the placenta, we performed immunofluorescent analysis. Similar to the positive control of single 8-cell blastomere (FIG. 12f), derivatives of TPS cells integrated into the trophoblast layers of the chimeric placentas and expressed the trophoblast markers CK8, MCT4 and TFAP2C (FIG. 3e and FIG. 12e), suggesting these chimeric cells can further differentiate into trophoblast lineages. As the negative control, we did not detect the expression of tdTomato in the non-injected placentas (FIG. 12g).

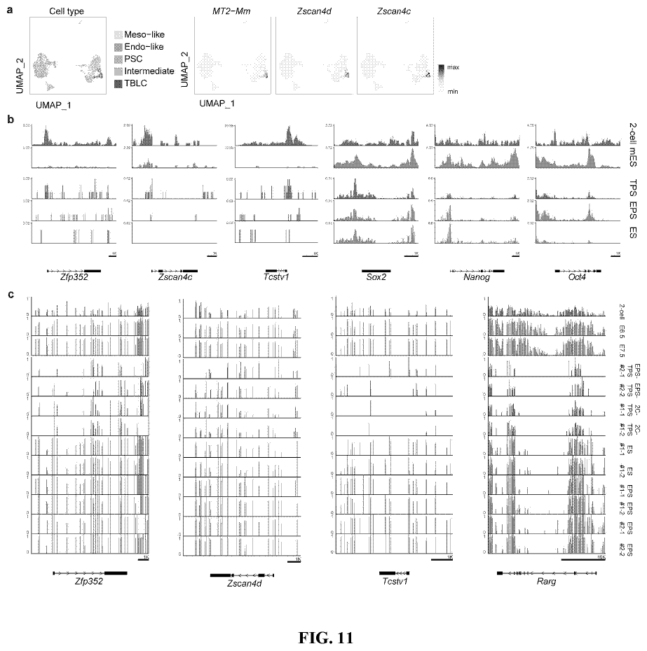

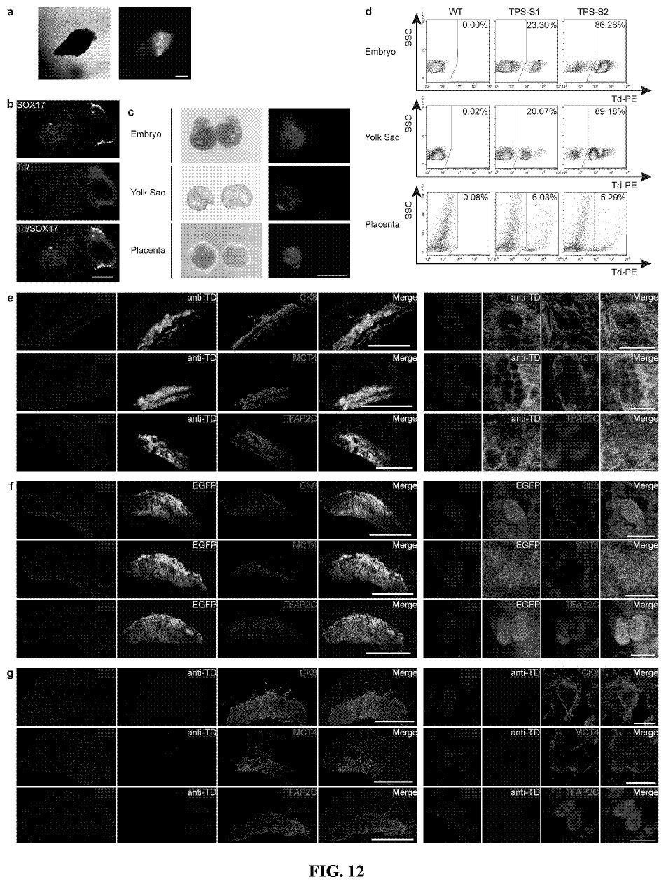

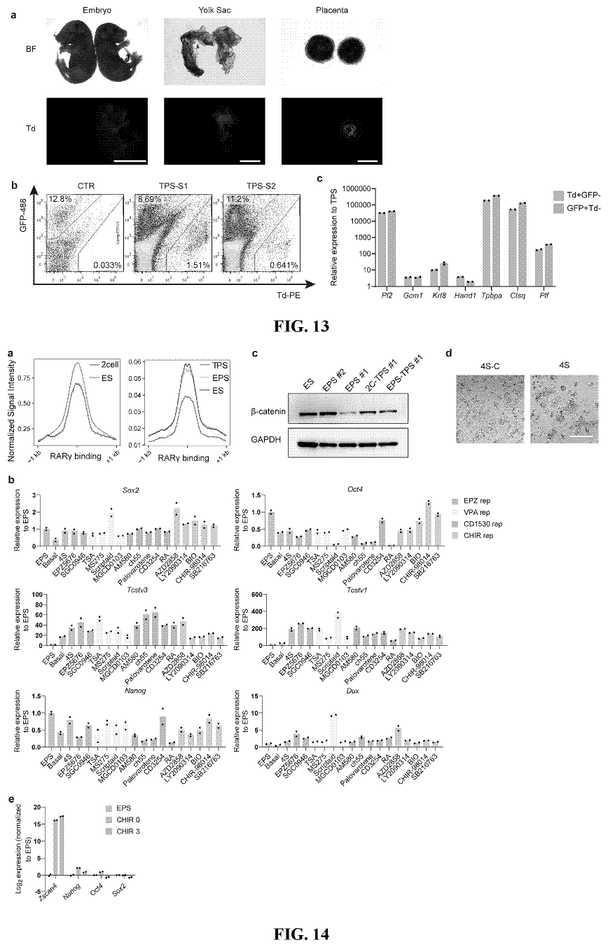

To further analyze the trophoblast identity of chimeric cells in the placenta at late developmental stages, we generated E17.5-E18.5 chimeric conceptuses using TPS cells (FIG. 13a). We performed single cell RNA sequencing analysis using cells from the chimeric E17.5 placenta (FIG. 3f). Among the 9,765 analyzed cells from the chimeric placenta, 1,924 cells expressed tdTomato (FIG. 3g), indicating that they are derivatives of TPS cells. Notably, these TPS derivatives contained extraembryonic lineages expressing trophoblast subtype markers such as Ctsq (trophoblast giant cells), Prl3a1 (spongiotrophoblasts), Pla2g4d (glycogen trophoblast cells), and Lgr5 (labyrinth trophoblast progenitor) (FIG. 3h). Moreover, these TPS-derived trophoblast cells are transcriptomically similar to their in vivo counterparts (FIG. 3f). These results were further supported by Q-PCR analysis using FACS-purified chimeric TPS derivatives from E18.5 placenta (FIG. 13b-c). To exclude the potential artifact of cell fusion in the placenta, we injected tdTomato reporter-labeled TPS cells into mouse 8-cell embryos that expressed GFP. Importantly, the majority of chimeric tdTomato-positive cells in the placenta did not show GFP signal (FIG. 13b-c). Collectively, the chimeric analyses provide strong evidence that TPS cells have extraembryonic and embryonic developmental potentials in vivo at the single cell level.

Induction of Blastocyst-Like Structures from TPS Cells In Vitro

Because TPS cells have embryonic and extraembryonic developmental potentials, we explored whether they can be induced into blastocyst-like structures (blastoids) in vitro. FGF, BMP and Yap signaling are important for preimplantation development especially for extraembryonic lineage development, and we attempted to treat TPS cells using bFGF, FGF4, BMP4 and LPA. TPS cells were seeded onto AggreWell microwell plate and treated using these factors, which generated small aggregates. Notably, these aggregates could further form cavity and morphologically resembled preimplantation blastocysts (FIG. 4a). Immunofluorescent analysis showed that blastoids induced from TPS cells expressed trophectoderm (CDX2) and epiblast (OCT4) markers (FIG. 4b).

To analyze the transcriptome features of TPS-blastoids, we performed single cell RNA sequencing analysis. Cluster analysis divided the analyzed 914 cells into 3 clusters, and these 3 clusters expressed markers of epiblast, primitive endoderm and trophectoderm respectively, including Oct4 (epiblast), Sox17 (primitive endoderm) and Gata2 (trophectoderm) (FIG. 4c). It is also notable that the expression of totipotent marker genes was nearly absent in the 3 clusters (FIG. 4c), suggesting that cells in the TPS-blastoids lose totipotent features. Next, a panel of 262 representative lineages markers of epiblast, primitive endoderm and trophectoderm were analyzed in the TPS-blastoid cells. Consistent with cluster analysis, the three clusters clustered well into epiblast, primitive endoderm and trophectoderm-like lineage respectively (FIG. 4d). We further compared the transcriptomic features of these TPS-blastoid cells with that of E4.5 blastocyst, and found that most of the TPS-blastoid cells clustered together with blastocyst cells assigned to the same lineage (FIG. 4e).

We further analyzed the transcriptomic similarity between TPS-blastoid cells and blastocyst cells. Epiblast cells from E4.5 to E7.5 and extraembryonic ectoderm cells from the post-implantation stages (E5.25 to E6.5) were also included in the analysis. As the control, we also analyzed blastoids generated by combining mouse TS cells with mouse ES or EPS cells (Rivron, N. C. et al. Blastocyst-like structures generated solely from stem cells. Nature 557, 106-111, doi:10.1038/s41586-018-0051-0 (2018); Sozen, B. et al. Self-Organization of Mouse Stem Cells into an Extended Potential Blastoid. Dev Cell 51, 698-712.e698, doi:10.1016/j.devcel.2019.11.014 (2019)). Importantly, cells resembling the epiblast, primitive endoderm and trophectoderm in the TPS-blastoids were transcriptionally similar to the three lineages from the E4.5 blastocyst stage (FIG. 4f). Notably, compared to trophectoderm-like cells from TPS-blastoids, there are significant transcriptomic differences between trophectoderm-like cells from previously reported blastoids and in vivo trophectoderm (FIG. 4f). These results were further supported by ssGSEA and quadratic programming-based identity score calculation (FIG. 4g-h). Collectively, these data suggest that TPS-blastoids contain the three lineages of blastocysts, which transcriptomically resemble E4.5 blastocyst.

We further evaluated the in vivo developmental potentials of TPS-blastoids. To this end, tdTomato reporter-labeled TPS-blastoids were transferred into pseudopregnant mice at 2.5 days post-coitum (dpc). At 6.5 dpc, decidualization was induced by TPS-blastoids (FIG. 4i-j), which was further evidenced by the induction of PTGS2 expression in the deciduae. These results suggest that TPS-blastoids can implant and trigger decidualization in vivo.

### Mechanistic Exploration of Totipotency Induction and Maintenance in TPS Cells

We attempted to explore the mechanism of regulating totipotency by the 4S condition. Because VPA, CD1530 and EPZ004777 showed synergistic effects in inducing totipotent marker gene expression, we first focused on analyzing the molecular targets of these three compounds. Among the 3 small molecules, VPA is reported to be an HDAC inhibitor, CD1530 an RARγ agonist, and EPZ004777 a Dot1L inhibitor. To analyze whether these small molecules indeed target these molecular targets in TPS cells, we first analyzed the levels of histone acetylation and H3K79 methylations in TPS cells by western blotting. Compared to that in EPS cells, we found that the level of histone H3 acetylation was increased whereas that of H3K79 methylation was reduced (FIG. 5a). In addition, upregulation of classical RAR downstream targets genes in TPS cells was observed (FIG. 5b). Interestingly, we found that the chromatin accessibility of RARγ binding loci in TPS cells is more closed when compared to that in EPS cells, and a similar result was also observed in 2-cell embryos (FIG. 14a).

To confirm that these small molecules indeed induce totipotency by targeting these reported targets, we first replaced VPA, EPZ004777 and CD1530 respectively with other small molecules targeting the same molecular targets, including HDAC, DOT1L, and RAR. We found that these replacements can also support the induction of totipotent marker genes (FIG. 5c and FIG. 14b). Consistent with these results, knockdown of hdac1/2 and Dot1l could also replace VPA and EPZ004777 in inducing totipotent markers respectively (FIG. 5d-g), and inhibition of RARγ signaling by small molecules greatly reduced the expression of totipotent markers during TPS induction and maintenance (FIG. 5h). Interestingly, Q-PCR analysis showed that the treatment of the 4 small molecules on preimplantation embryos could facilitate the maintenance of totipotent marker gene expression in preimplantation mouse embryos beyond the 2-cell embryo stage (FIG. 5i). Taken together, these data suggest that inhibition of HDAC1/2 and DOT1L activity and activation of RARγ signaling are important for inducing totipotency in TPS cells.

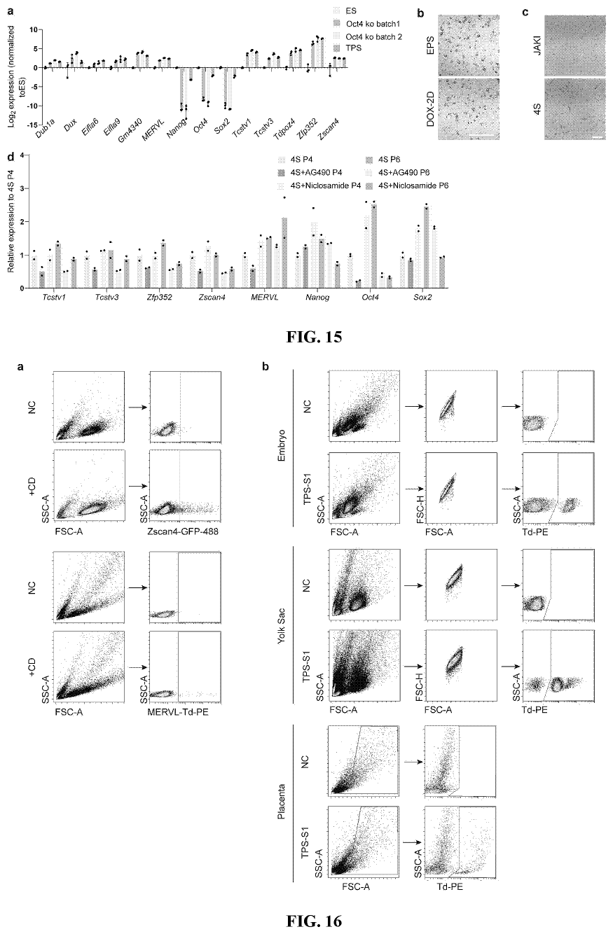

Next, we sought to analyze the role of CHIR 99021 in the induction and maintenance of TPS cells. Consistent with its reported roles of activating Wnt signaling by inhibiting GSK3p, we found that TPS cells expressed β-catenin at the protein level (FIG. 14c). To examine whether CHIR 99021 is required for maintaining TPS cells, we removed CHIR 99021 from the chemical cocktail during culturing TPS cells. The omission of CHIR 99021 led to reduced proliferation during the first few passages (about 3-5 passages). After further passaging, we found that CHIR 99021 is not required for the proliferation of TPS cells (FIG. 14d). Q-PCR analysis further showed that the expression of totipotent marker genes in TPS cells is not affected by the omission of CHIR 99021 after long-term culturing (FIG. 14e). However, we also found that the omission of CHIR 99021 can greatly reduce the in vivo chimeric of TPS cells (Table 2). Therefore, CHIR 99021 is beneficial for promoting proliferation during TPS conversion and important for maintaining the in vivo developmental potency of TPS cells. Finally, we attempted to explore the roles of known key totipotent and pluripotent regulators in the induction and maintenance of TPS cells. Dux is an important regulator for inducing 2C-like cells, and p53 is reported to be a key upstream activator of Dux in inducing 2C-like cells. During the maintenance of TPS cells, we found that knockdown of Dux expression led to downregulation of several totipotent markers (MERVL, Zfp352, Tcstv3), whereas expression of other totipotent markers (Tcstv1, Zscan4) was not significantly affected (FIG. 5j-k). On the other hand, p53 knockdown did not cause decreased expression of totipotent markers during the induction of TPS cells (FIG. 5l). Next, we examined the role of Oct4 and LIF signaling in the maintenance of TPS cells, which are key pluripotent regulators. Knockout of Oct4 in TPS cells did not significantly affect the expression of totipotent marker genes (FIG. 15a), whereas the proliferation of TPS cells was reduced (FIG. 15b). Similar to Oct4, inhibition of LIF signaling by small molecules also led to an impaired proliferation of TPS cells (FIG. 15c), and the expression of totipotent markers was not affected (FIG. 15d). Collectively, these data suggest that the mechanism of totipotency regulation in TPS cells is unique and distinct from that of 2C-like cells, which requires further exploration.

### DISCUSSION

It is challenging to derive totipotent stem cells in vitro that functionally and molecularly resemble cells from totipotent embryos. Here, we report that a chemical cocktail enables the derivation of totipotent-like stem cells, designated as totipotent potential stem (TPS) cells, from 2-cell mouse embryos and extended pluripotent stem cells that can be stably maintained long-term in vitro. TPS cells shared features with 2-cell mouse embryos in terms of totipotent markers, transcriptome, chromatin accessibility and DNA methylation patterns. In vivo chimeric assays show that these cells have embryonic and extraembryonic developmental potentials at the single cell level. Moreover, we show that TPS cells can be induced into blastocyst-like structures resembling preimplantation mouse blastocysts. Mechanistically, inhibition of HDAC1/2 and DOT1L activity and activation of RARγ signaling are important for inducing and maintaining totipotent features of TPS cells. Our study demonstrates the feasibility of capturing and maintaining totipotency in vitro.

In this study, TPS cells showed transcriptomic and epigenetic features that resemble 2-cell mouse embryos. Importantly, TPS cells have the capacity to generate both embryonic and extraembryonic lineages in vivo at the single cell level. When self-organizing in vitro, TPS cells can form blastocyst-like structures, which transcriptomically resemble E4.5 blastocysts and can implant and trigger decidualization in vivo. Mechanistic studies showed that HDAC1/2 and DOTIL inhibition as well as RARγ signaling activation are important for inducing and maintaining totipotent features of TPS cells. These results demonstrate the feasibility of capturing and maintaining totipotent-like stem cells in vitro.

The derivation of TPS cells from 2-cell mouse embryos represents an important step toward capturing authentic totipotency in vitro. A long-standing scientific question in the field of stem cells is whether totipotent stem cells can be captured from totipotent embryos, which has not been achieved. To date, efforts to generate totipotent-like cells, including 2C-like cells and TBLCs rely solely on the conversion from pluripotent stem cells and have not been validated on totipotent embryos. In contrast, our results showed that the treatment of the 4 small molecules on preimplantation embryos could facilitate the maintenance of totipotent marker gene expression in preimplantation mouse embryos beyond the 2-cell embryo stage (FIG. 5i). Moreover, the 4S condition permitted establishing 2-cell embryo-derived TPS cells, which highly expressed totipotency marker genes (FIG. 1c), shared transcriptomic and epigenetic features with 2-cell embryos (FIG. 2), and have bi-directional developmental potentials which permits its ability to form blastoids (FIG. 3e and FIG. 4). These findings provide the proof-of-principle evidence that it is possible to directly derive totipotent-like stem cells from totipotent embryos, which represents an important step toward capturing bona fide 2-cell totipotent embryos in vitro. In this regard, TPS cells directly derived from 2-cell embryos could represent the in vitro model that is closest to in vivo totipotent embryos up-to-date. Another key unique feature of TPS cells is their ability to self-organize to generate blastocyst-like structures, which mimic the natural developmental process. Upon the stimulation of natural signaling molecules that drive early preimplantation development, TPS cells can self-organize into blastoids which transcriptomically resemble in vivo E4.5 blastocyst (FIG. 4a-g). Moreover, these blastoids can implant and induce decidualization in vivo (FIG. 4i-j). Previous efforts on generating synthetic blastocysts using pluripotent cells rely on the combined use of TS cells or reprogramming from epiblast stem cells. The major limitation of using TS cells is that they transcriptionally resemble postimplantation extraembryonic ectoderm (ExE) but not preimplantation trophectoderm, therefore the induction process is artificial and unnatural, which led to the significant transcriptomic differences between in vivo trophectoderm and TS-derived trophectoderm-like cells from the blastoids (FIG. 4f-h). Likewise, induction of blastocyst-like cysts (iBLCs) from epiblast stem cells by reprogramming does not mimic the natural developmental process. In contrast, the generation of TPS-blastoids relied on the differentiation of totipotent-like stem cells to the blastocyst lineages (FIG. 4c), which more closely resembles the process of blastocyst formation in vivo. On the other hand, unlike EPS-blastoids that contain a significant portion of intermediates and mesodermal cells, the majority of TPS-blastoids contained the three blastocyst lineages and intermediates were nearly absent (FIG. 4c-d). Therefore, TPS-blastoids avoid the limitations of previously reported synthetic blastocysts and could serve as a powerful model to recapitulate the in vivo process of blastocyst formation in vitro. It is also important to further optimize the TPS-based blastoid induction approach and explore whether they can develop into conceptuses in vivo.

Finally, TPS cells provide a valuable in vitro platform to explore the regulation of totipotency, and clarify the molecular targets of the small molecules used in the 4S condition could provide a novel mechanistic insight into the regulation of totipotency. Importantly, we found that inhibition of HDAC1/2 and DOT1L activity and activation of RARγ signaling are important for inducing totipotency in TPS cells (FIG. 5a-h). Interestingly, the role of RARγ signaling in totipotency regulation is supported by a recent study showing that RA signaling is critical during the totipotent window in early development. It would be important to further explore the synergetic effect of these targets in maintaining totipotency in embryos. Recent studies suggest that 2C-like cells do not fully recapitulate two-cell embryos in terms of regulation of two-cell embryo-specific genes and cautions should be taken when studying totipotency using 2C-like cells as the model system, highlighting the demand for establishing new in vitro model systems for studying totipotency. Although one recent study showed the induction of TBLCs by spliceosome inhibition, this approach only enabled their conversion from pluripotent stem cells but not capturing from totipotent embryos, whereas our 4S condition permits deriving TPS cells from 2C embryos. In addition, in contrast to spliceosome inhibition that broadly affect gene expression in a non-specific manner, inducing and maintaining totipotency by 4S condition relies on the combined effect of regulating specific signaling pathways and epigenetic regulators (FIG. 5a-h), which is more specific to regulate totipotency. Indeed, the transcriptomic profiles of the TPS 2C-subpopulation are significantly closer to that of middle-to-late 2C embryos when compared to that of TBLCs (FIG. 2b-d). Moreover, our 4S condition also bypasses potential safety concerns associated with spliceosome inhibition, such as tumor induction or proliferation difficulty. Therefore, TPS cells have wide applicative potentials as a new platform favorable for studying totipotency in vitro.

In summary, our study demonstrates the feasibility of capturing totipotent stem cells from early embryos. TPS cells would provide a useful tool for studying the mechanism of totipotency regulation as well as early preimplantation embryogenesis. Our study also opens up a path toward capturing totipotent stem cells from other mammalian species including humans.

1. A cell culture media composition for deriving cell totipotency in vitro of isolated cells, the composition comprising chemical derivers of totipotency (CDTs) from each of the following groups
(1) an HDAC inhibitor,
(2) a Dot1L inhibitor,
(3) an RARγ agonist, and
(4) optionally, a GSK inhibitor,
in amounts effective to induce an untreated cell into a totipotent potential stem (TPS) cell.

2. The composition of claim 1, wherein;
(i) the HDAC inhibitor is an Hdac1 and/or Hdac2 inhibitor, or is selected from the group consisting of VPA (“V”), TSA, MS275, Scrptaid, SAHA, LBH589, FK228, PXD101, Sodium butyrate, LAQ824, CUDC-101, JNJ-26481585, SB939, PCI-24781, ACY-1215, CI994, CUDC-907, RGFP109, Resminostat, Curcumin, Divalproex Sodium, 4-PBA, GSK3117391, CAY10433, CM-675 and MGCD0103;
(ii) the Dot1L inhibitor is selected from the group consisting of EPZ004777 (“E”), EPZ5676, and SGC0946;
(iii) the RARγ agonist is selected from the group consisting of CD1530 (“D”), AM580, ch55, Palovarotene, CD3254, CD5789, CD437, TTNPB, AGN 205327 and RA; and/or
(iv) the GSK inhibitor is a GSK3 inhibitor, or is selected from the group consisting of CHIR99021 (“C”), AZD2858, LY2090314, BIO, CHIR 98014, SB415286, AZD1080, BRD3731, A 1070722, BIP-135 and SB216763.

3-7. (canceled)

8. The composition of claim 1, further comprising VPA (“V”), EPZ004777 (“E”), and CD1530 (“D”), wherein V is in a concentration range from 10-1000 μM; E is in a concentration range from 0.1-10 μM; and D is in a concentration range from 0.1-5 μM.

9. The composition of claim 8, further comprising CHIR99021 (“C”), wherein C is in a concentration range from 0.5-10 μM.

10. The composition of claim 1, wherein the composition is in a kit, wherein the CDTs are present in relative amounts to put into cell culture media for pluripotent cells to induce totipotency.

11. A method of inducing totipotency in donor cells, the method comprising: culturing the donor cells or cells derived therefrom with the composition of claim 1 for a period of time effective to induce totipotency.

12. The method of claim 11, wherein the donor cells or cells derived therefrom are selected from the group consisting of cells isolated from a non-human embryo, embryonic stem cells, induced pluripotent stem cells, extended pluripotent stem cells, multipotent stem cells, cells of hematological origin, cells of embryonic origin, skin derived cells, fibroblasts, adipose cells, epithelial cells, endothelial cells, mesenchymal cells, parenchymal cells, neurological cells, and connective tissue cells.

13. The method of claim 11, wherein the donor cells or cells derived therefrom are selected from the group consisting of mouse embryonic stem cells and induced pluripotent stem cells.

14. The method of claim 11, wherein the donor cells or cells derived therefrom are selected from the group consisting of cells isolated from 2-cell mouse embryo and extended pluripotent stem cells.

15. The method of claim 11, wherein the donor cells or cells derived therefrom are firstly induced into extended pluripotent stem cells, and then the extended pluripotent stem cells are cultured with the composition.

16. The method of claim 11, wherein the donor cells are seeded as single cells or as small colonies.

17. An isolated chemically induced totipotent potential stem cell (ciTPSC), with totipotency, when compared to a corresponding cell, or an isolated cell population comprising at least 10% of the isolated cells.

18. An isolated chemically induced totipotent potential stem cell (ciTPSC) obtained by the method of claim 11.

19. The isolated ciTPSC of claim 18, wherein, i) the cell expression of any one or more totipotent marker gene selected from Zscan4, Zfp352, Tcstv1, Tcstv3, MERVL, Dux, Dubla, Eif1al6, Eif1al9, Gm4340 and Tdpoz4 is present when compared to untreated corresponding cells, optionally after 5 or more passages, and/or ii) the cell expression of any one or more pluripotency marker gene selected from Oct4, Nanog and Sox2 is downregulated when compared to untreated corresponding cells.

20. The isolated ciTPSC of claim 18, wherein the cell can generate both embryonic and extraembryonic lineages at a single cell level.

21. The isolated ciTPSC of claim 18, wherein the cell can form blastocyst-like structures in vitro.

22. A therapeutic composition comprising the isolated ciTPSC of claim 18, formulated for administration to an individual by injection, implantation of a prosthetic device or tissue engineering matrix.

23. A cell culture, comprising: the isolated ciTPSC of claim 18, and a culture medium comprising (1) an HDAC inhibitor, (2) a Dot1L inhibitor, (3) an RARγ agonist, and (4) optionally, a GSK inhibitor.

24. The cell culture of claim 23, wherein said culture medium is effectively maintains the isolated ciTPSC in a totipotent state for at least 2, 3, 4, 5, 6, 7, 8, 9, 10, 11, 12, 13, 14, or 15 passages.

25. The cell culture of claim 24, wherein the isolated ciTPSC maintain expression of a totipotent marker selected from Zscan4, Zfp352, Tcstv1, Tcstv3, MERVL, Dux, Dub1a, Eif1al6, Eif1a9, Gm4340 and Tdpoz4.
